(19) United States
(12) Patent Application Publication
(10) Pub. No.: US 2024/0307120 A1
(43) Pub. Date: Sep. 19, 2024

(54) MULTI-STAGE PATIENT-SPECIFIC SURGICAL PLANS AND SYSTEMS AND METHODS FOR CREATING AND IMPLEMENTING THE SAME

(71) Applicant: Carlsmed, Inc., Carlsbad, CA (US)

(72) Inventors: Niall Patrick Casey, Carlsbad, CA (US); Michael J. Cordonnier, Carlsbad, CA (US); Syed Shariq Hussain, Vista, CA (US)

(21) Appl. No.: 18/120,979

(22) Filed: Mar. 13, 2023

(51) Int. Cl. A61B 34/10; G16H 10/60; G16H 20/40

(52) U.S. Cl. CPC A61B 34/10; G16H 10/60; G16H 20/40; A61B 2034/102; A61B 2034/107

(57) ABSTRACT

Systems and methods for determining and implementing multi-stage, patient-specific surgical plans are described herein. The multi-stage treatment plans can include a comprehensive overview of specific surgical interventions to be performed over a period of time (e.g., 5 years, 10 years, 20 years, etc.). The surgical interventions can be specifically selected, timed, and sequenced to provide a higher likelihood of achieving favorable patient outcomes and improved quality of life.

### TECHNICAL FIELD

The present disclosure is generally related to designing and implementing medical care, and more particularly to systems and methods for designing and implementing patient-specific surgical procedures and/or medical devices.

### BACKGROUND

Surgical procedures to implant orthopedic implants are used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include osteoarthritis, lumbar degenerative disc disease or cervical degenerative disc disease, lumbar spinal stenosis, and cervical spinal stenosis.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a network connection diagram illustrating a system for providing patient-specific medical care in accordance with embodiments of the present technology.

FIG. 2 illustrates a computing device suitable for use in connection with the system of FIG. 1 in accordance with embodiments of the present technology.

FIG. 3 is a flow diagram illustrating a method for providing patient-specific medical care in accordance with embodiments of the present technology.

FIG. 4 is a flow diagram illustrating a method for determining a multi-stage patient-specific surgical plan in accordance with embodiments of the present technology.

FIGS. 5A and 5B illustrate a representative multi-stage surgical plan generated in accordance with embodiments of the present technology.

FIG. 6 is a flow diagram illustrating another method for providing patient-specific medical care in accordance with embodiments of the present technology.

FIGS. 7A-7C illustrate representative data sets that may be used and/or generated in connection with the methods described herein, in accordance with embodiments of the present technology.

FIG. 8 is a flow diagram illustrating another method for providing patient-specific medical care in accordance with embodiments of the present technology.

FIG. 9 is a partially schematic illustration of an operative setup and associated computing systems for providing patient-specific medical care in accordance with embodiments of the present technology.

FIGS. 10A and 10B illustrate a representative patient-specific implant that can be used and/or generated in connection with the methods described herein in accordance with embodiments of the present technology.

FIG. 11 illustrates a segment of a patient's spine after several patient-specific implants have been implanted therein in accordance with embodiments of the present technology.

### DETAILED DESCRIPTION

The present technology is directed to systems and methods for planning and implementing medical procedures and/or devices. For example, in many of the embodiments disclosed herein, a method of providing medical care includes generating a multi-stage, patient-specific medical treatment plan for a patient. The multi-stage treatment plans can include a comprehensive overview of specific surgical interventions to be performed over a period of time (e.g., 5 years, 10 years, 20 years, etc.). The surgical interventions can be specifically selected, timed, and sequenced to provide a higher likelihood of achieving favorable patient outcomes and improved quality of life. As an added benefit, because the multi-stage plans are “prospective,” the plans enable a patient, caregiver, surgeon, and/or other healthcare provider to better understand long-term treatment plans and likely outcomes for a particular patient before a first surgery is performed.

The multi-stage treatment plans described herein can include a first treatment stage that includes a first surgical procedure at a first target location, and a second treatment stage that includes a second surgical procedure at a second target location. The second surgical procedure is generally based at least in part on the first surgical procedure, and/or vice versa. For example, the second surgical procedure may be less invasive or involved than if the patient had elected to not undergo the first procedure. The second surgical procedure may also be based on a patient's predicted long-term response to the first surgical procedure. The first and second treatment stages are spaced apart in time by either a fixed or adaptive duration, as described in detail below, but typically are separated in time by at least 6 months.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

The headings are provided for convenience only and should not be used to interpret the scope of the present technology.

### A. Select Embodiments of Systems for Designing Patient-Specific Surgical Plans and Patient-Specific Implants

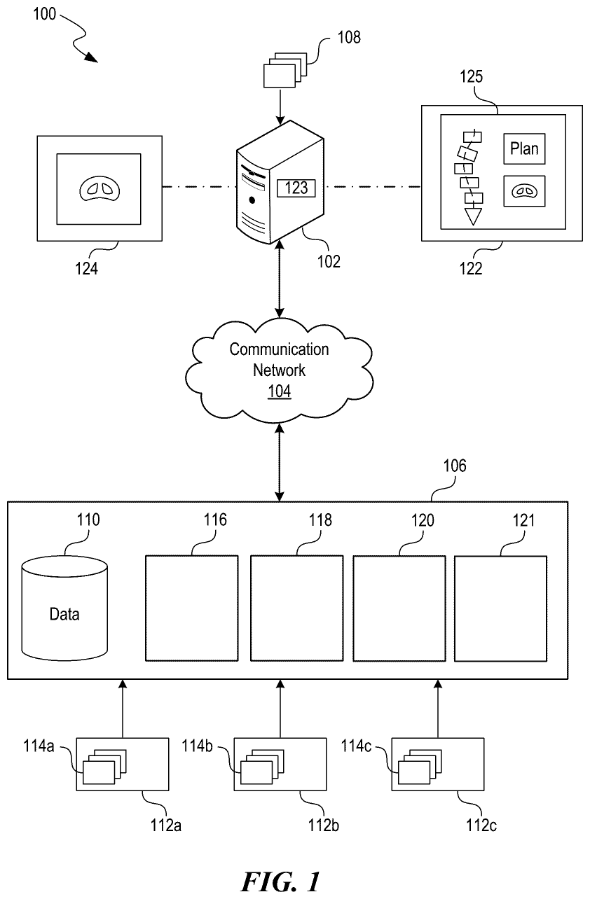

FIG. 1 is a network connection diagram illustrating a system 100 for providing patient-specific medical care, according to embodiments of the present technology. As described in further detail herein, the system 100 is configured to generate a multi-stag medical treatment plan for a patient. In some embodiments, the system 100 is configured to generate a medical treatment plan for a patient suffering from an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, or cervical spinal stenosis, or a combination thereof. The medical treatment plan can include surgical information, technology recommendations (e.g., device and/or instrument recommendations), and/or medical device designs. For example, the multi-stage medical treatment plan can include two or more surgical procedures (e.g., a surgical procedure or intervention) to be performed in a specific sequence with a specific temporal separation. Each surgical procedure may also include at least one medical device (e.g., an implanted medical device (also referred to herein as an “implant” or “implanted device”) or implant delivery instrument) for use with each stage of the multi-stage medical procedure. In some embodiments, the medical treatment plan is therefore also referred to as a “multi-stage treatment plan,” a “multi-stage surgical plan,” “surgical plan,” a “patient-specific surgical plan,” a “patient-specific treatment plan,” or the like.

In some embodiments, the system 100 generates a multi-stage medical treatment plan that is customized for a particular patient or group of patients, also referred to herein as a “patient-specific” or “personalized” treatment or surgical plan. The patient-specific surgical plan can include at least one patient-specific surgical procedure and/or at least one patient-specific medical device that are designed and/or optimized for the patient's particular characteristics (e.g., condition, anatomy, pathology, condition, medical history). For example, the patient-specific medical device can be designed and manufactured specifically for the particular patient, rather than being an off-the-shelf device. However, it shall be appreciated that a patient-specific surgical plan can also include aspects that are not customized for the particular patient. For example, a patient-specific or personalized surgical procedure can include one or more instructions, portions, steps, etc. that are non-patient-specific. Likewise, a patient-specific or personalized medical device can include one or more components that are non-patient-specific, and/or can be used with an instrument or tool that is non-patient-specific. Personalized implant designs can be used to manufacture or select patient-specific technologies, including medical devices, instruments, and/or surgical kits. For example, a personalized surgical kit can include one or more patient-specific devices, patient-specific instruments, non-patient-specific technology (e.g., standard instruments, devices, etc.), instructions for use, patient-specific treatment plan information, or a combination thereof.

The system 100 includes a client computing device 102, which can be a user device, such as a smart phone, mobile device, laptop, desktop, personal computer, tablet, phablet, or other such devices known in the art. As discussed further herein, the client computing device 102 can include one or more processors, and memory storing instructions executable by the one or more processors to perform the methods described herein. The client computing device 102 can be associated with a healthcare provider (e.g., a surgeon, healthcare administrator, hospital system, etc.) that is treating the patient. Although FIG. 1 illustrates a single client computing device 102, in alternative embodiments, the client computing device 102 can instead be implemented as a client computing system encompassing a plurality of computing devices, such that the operations described herein with respect to the client computing device 102 can instead be performed by the client computing system and/or the plurality of client computing devices.

The client computing device 102 is configured to receive a patient data set 108 associated with a patient to be treated. The patient data set 108 can include data representative of the patient's condition, anatomy, pathology, medical history, preferences, and/or any other information or parameters relevant to the patient. For example, the patient data set 108 can include medical history, surgical intervention data, treatment outcome data, progress data (e.g., physician notes), patient feedback (e.g., feedback acquired using quality of life questionnaires, surveys), clinical data, provider information (e.g., physician, hospital, surgical team), patient information (e.g., demographics, sex, age, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), or the like. In some embodiments, the patient data set 108 includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine.

The client computing device 102 is also configured to enable a user (e.g., a surgeon) to review one or more multi-stage surgical plans for a patient to be treated. In particular, the client computing device 102 can include a surgical plan review software module 123 (“the review module 123”). The review module 123 can comprise computer-executable instructions for generating, displaying, and/or implementing a surgical plan review program or platform 125 (“the review program 125”) that facilitates surgeon or user review of one or more patient-specific multi-stage surgical plans via the client computing device 102.

The review module 123 can be stored in the form of computer-readable or computer-executable instructions on a memory (not shown) of the client computing device 102. In other embodiments, the review module 123 can be stored remotely from the client computing device 102 (e.g., in the cloud or at a remote server) and implemented on the client computing device 102 via a remote (e.g., wireless) connection. In yet other embodiments, some of the review module 123 can be stored locally at the client computing device 102 while other aspects of the review module 123 can be store remotely.

The client computing device 102 is operably connected via a communication network 104 to a server 106, thus allowing for data transfer between the client computing device 102 and the server 106. The communication network 104 may be a wired and/or a wireless network. The communication network 104, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 106, which may also be referred to as a “treatment assistance network” or “prescriptive analytics network,” can include one or more computing devices and/or systems. As discussed further herein, the server 106 can include one or more processors, and memory storing instructions executable by the one or more processors to perform some or all of the methods described herein. In some embodiments, the server 106 is implemented as a distributed “cloud” computing system or facility across any suitable combination of hardware and/or virtual computing resources.

The client computing device 102 and server 106 can individually or collectively perform some or all of the various methods described herein for providing patient-specific medical care. For example, some or all of the steps of the methods described herein can be performed by the client computing device 102 alone, the server 106 alone, or a combination of the client computing device 102 and the server 106. Thus, although certain operations are described herein with respect to the server 106, it shall be appreciated that these operations can also be performed by the client computing device 102, and vice-versa, unless the context clearly dictates otherwise.

The server 106 includes at least one database 110 configured to store reference data useful for the treatment planning methods described herein. The reference data can include historical and/or clinical data from the same or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 110 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient's condition, anatomy, pathology, medical history, disease progression, preferences, and/or any other information or parameters relevant to the reference patient, such as any of the data described herein with respect to the patient data set 108. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, and/or treatment level of the spine.

As another example, a reference patient data set can include treatment data regarding at least one surgical procedure performed on the reference patient, such as descriptions of surgical procedures or interventions (e.g., surgical approaches, bony resections, surgical maneuvers, corrective maneuvers, placement of implants or other devices). The treatment data can further include whether additional surgical procedures or interventions were necessary following the initial surgical procedure, what the additional surgical procedures or interventions were, the timing of the additional surgical procedures or interventions, and scored outcomes from the additional surgical procedures or interventions. The treatment data can also include medical device design data for at least one medical device used to treat the reference patient (e.g., medical devices implanted during the surgical procedure or intervention), such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). The reference patient data set can also include outcome data representing an outcome of the treatment of the reference patient, such as corrected anatomical metrics, presence of fusion, HRQL, pain level, activity level, return to work, complications, recovery times, efficacy, mortality, and/or follow-up surgeries.

In some embodiments, the server 106 receives at least some of the reference patient data sets from a plurality of healthcare provider computing systems (e.g., systems 112a-112c, collectively 112). The server 106 can be connected to the healthcare provider computing systems 112 via one or more communication networks (not shown). Each healthcare provider computing system 112 can be associated with a corresponding healthcare provider (e.g., physician, surgeon, medical clinic, hospital, healthcare network, etc.). Each healthcare provider computing system 112 can include at least one reference patient data set (e.g., reference patient data sets 114a-114c, collectively 114) associated with reference patients treated by the corresponding healthcare provider. The reference patient data sets 114 can include, for example, electronic medical records, electronic health records, biomedical data sets, etc. The reference patient data sets 114 can be received by the server 106 from the healthcare provider computing systems 112 and can be reformatted into different formats for storage in the database 110. Optionally, the reference patient data sets 114 can be processed (e.g., cleaned) to ensure that the represented patient parameters are likely to be useful in the treatment planning methods described herein.

As described in further detail herein, the server 106 can be configured with one or more algorithms that generate multi-stage patient-specific surgical plan data (e.g., treatment procedures, target anatomical corrections, medical devices, etc.) based on the reference data. In some embodiments, the patient-specific data is generated based on correlations between the patient data set 108 and the reference data. Optionally, the server 106 can predict outcomes, including recovery times, efficacy based on clinical end points, likelihood of success, predicted mortality, predicted related follow-up surgeries, or the like. In some embodiments, the server 106 can continuously or periodically analyze patient data (including patient data obtained during the patient stay) to determine near real-time or real-time risk scores, mortality prediction, etc.

In some embodiments, the server 106 includes one or more modules for performing one or more steps of the patient-specific treatment planning methods described herein. For example, in the depicted embodiment, the server 106 includes a data analysis module 116, a treatment planning module 118, a disease progression module 120, and an intervention timing module 121. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments.

The data analysis module 116 is configured with one or more algorithms for identifying a subset of reference data from the database 110 that is likely to be useful in developing a patient-specific treatment plan. For example, the data analysis module 116 can compare patient-specific data (e.g., the patient data set 108 received from the client computing device 102) to the reference data from the database 110 (e.g., the reference patient data sets) to identify similar data (e.g., one or more similar patient data sets in the reference patient data sets). The comparison can be based on one or more parameters, such as age, gender, BMI, lumbar lordosis, pelvic incidence, and/or treatment levels. The parameter(s) can be used to calculate a similarity score for each reference patient. The similarity score can represent a statistical correlation between the patient data set 108 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients.

The data analysis module 116 can further be configured with one or more algorithms to select a subset of the reference patient data sets, e.g., based on similarity to the patient data set 108 and/or treatment outcome of the corresponding reference patient. For example, the data analysis module 116 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, presence of fusion, HRQL, activity level, complications, recovery times, efficacy, mortality, or follow-up surgeries. As described in further detail below, in some embodiments, the data analysis module 116 calculates an outcome score by assigning values to each outcome parameter. A patient can be considered to have a favorable outcome if the outcome score is above, below, or at a specified threshold value.

In some embodiments, the data analysis module 116 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 116 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular health-care provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific surgical plan to perform a battlefield surgery can be based on reference patient data from similar battlefield surgeries and/or datasets associated with battlefield surgeries. In another example, the patient-specific surgical plan can be generated based on available robotic surgical systems. The reference patient data sets can be selected based on patients that have been operated on using comparable robotic surgical systems under similar conditions (e.g., size and capabilities of surgical teams, hospital resources, etc.).

The treatment planning module 118 is configured with one or more algorithms to generate at least one surgical plan (e.g., pre-operative plans, intra-operative plans, post-operative plans etc.) based on the output from the data analysis module 116. In some embodiments, the treatment planning module 118 is configured to develop and/or implement at least one predictive model for generating the patient-specific treatment plan, also known as a “prescriptive model.” The predictive model(s) can be developed using clinical knowledge, statistics, machine learning, AI, neural networks, or the like. In some embodiments, the output from the data analysis module 116 is analyzed (e.g., using statistics, machine learning, neural networks, AI) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a surgical plan will produce a favorable outcome for the particular patient. The predictive model(s) can be validated, e.g., by inputting data into the model(s) and comparing the output of the model to the expected output.

In some embodiments, the treatment planning module 118 is configured to generate the surgical plan based on previous treatment data from reference patients. For example, the treatment planning module 118 can receive a selected subset of reference patient data sets and/or similar patient data sets from the data analysis module 116, and determine or identify treatment data from the selected subset. The treatment data can include, for example, treatment procedure data (e.g., surgical procedure or intervention data) and/or medical device design data (e.g. implant design data) that are associated with favorable or desired treatment outcomes for the corresponding patient. The treatment planning module 118 can analyze the treatment procedure data and/or medical device design data to determine an optimal treatment protocol for the patient to be treated. For example, the treatment procedures and/or medical device designs can be assigned values and aggregated to produce a treatment score. The multi-stage, patient-specific surgical plan can be determined by selecting surgical plan(s) based on the score (e.g., higher or highest score; lower or lowest score; score that is above, below, or at a specified threshold value). The personalized patient-specific surgical plan can be based on, at least in part, the patient-specific technologies or patient-specific selected technology.

Alternatively or in combination, the treatment planning module 118 can generate the surgical plan based on correlations between data sets. For example, the treatment planning module 118 can correlate treatment procedure data and/or medical device design data from similar patients with favorable outcomes (e.g., as identified by the data analysis module 116). Correlation analysis can include transforming correlation coefficient values to values or scores. The values/scores can be aggregated, filtered, or otherwise analyzed to determine one or more statistical significances. These correlations can be used to determine surgical procedure(s) and/or medical device design(s) that are optimal or likely to produce a favorable outcome for the patient to be treated.

Alternatively or in combination, the treatment planning module 118 can generate the surgical plan using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 118 generates the surgical plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 110, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training dataset can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate a surgical plan, the patient data set 108 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient. Based on these calculations, the trained machine learning model(s) can select at least one surgical plan for the patient. In some embodiments, the trained machine learning model(s) can determine candidate procedures (or candidate surgical plans), analyze the candidate procedures, select the candidate surgical plans or portions thereof, score plans, and/or generate surgical plans for the patient. Each surgical plan can be scored (e.g., scored based on favorable outcome, likelihood of outcome, etc.) and ranked according to the score. The trained machine learning model(s) can determine a set of surgical plans that meet selection criteria for plan review by a user. The selection criteria can be based on, for example, regulatory requirements, reimbursement criteria, healthcare/provider expertise, available surgical equipment, manufacturing capabilities, elimination criteria, combinations thereof, or the like. A user can input one or more selection criteria to control the types and/or features of the surgical plans for comparison. In embodiments where multiple trained machine learning models are used, the models can be run sequentially or concurrently to compare outcomes and can be periodically updated using training data sets. The treatment planning module 118 can use one or more of the machine learning models based the model's predicted accuracy score.

The patient-specific surgical plan generated by the treatment planning module 118 can include a multi-stage surgical plans. For example, as described in greater detail below, multi-stage plans described herein can include a first treatment stage that includes a first surgical procedure at a first target location, and a second treatment stage that includes a second surgical procedure at a second target location. The first surgical procedure is generally based at least in part on the first surgical procedure, and/or vice versa. For example, the second surgical procedure may be less intrusive or involved than if the patient had elected to not undergo the first procedure. The first and second treatment stages are spaced apart in time by either a fixed or adaptive duration, as described in detail below, but typically are separated in time by at least 6 months. Both the first and the second surgical procedures can include at least one patient-specific medical device (e.g., an implant or implant delivery instrument) to be implanted during and/or used to perform the corresponding surgical procedure. A patient-specific surgical plan can include an entire surgical procedure or portions thereof. Additionally, one or more patient-specific medical devices can be specifically selected or designed for the corresponding surgical procedure, thus allowing for the various components of the patient-specific technology to be used in combination to treat the patient.

In some embodiments, the patient-specific surgical procedure (e.g., the first and/or second surgical procedures for multi-stage surgical plans) includes an orthopedic surgery procedure, such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). In some embodiments, the patient-specific treatment procedure includes descriptions of and/or instructions for performing one or more aspects of a patient-specific surgical procedure. For example, the patient-specific surgical procedure can include one or more of a surgical approach, a corrective maneuver, a bony resection, or implant placement.

In some embodiments, the patient-specific medical device design includes a design for an orthopedic implant and/or a design for an instrument for delivering an orthopedic implant. Examples of such implants include, but are not limited to, screws (e.g., bone screws, spinal screws, pedicle screws, facet screws), interbody implant devices (e.g., intervertebral implants), cages, plates, rods, disks, fusion devices, spacers, rods, expandable devices, stents, brackets, ties, scaffolds, fixation device, anchors, nuts, bolts, rivets, connectors, tethers, fasteners, joint replacements, hip implants, or the like. Examples of instruments include, but are not limited to, screw guides, cannulas, ports, catheters, insertion tools, or the like.

A patient-specific medical device design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of a corresponding medical device. For example, a design for an orthopedic implant can include implant shape, size, material, and/or effective stiffness (e.g., lattice density, number of struts, location of struts, etc.). In some embodiments, the generated patient-specific medical device design is a design for an entire device. Alternatively, the generated design can be for one or more components of a device, rather than the entire device.

In some embodiments, the design is for one or more patient-specific device components that can be used with standard, off-the-shelf components. For example, in a spinal surgery, a pedicle screw kit can include both standard components and patient-specific customized components. In some embodiments, the generated design is for a patient-specific medical device that can be used with a standard, off-the-shelf delivery instrument. For example, the implants (e.g., screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The patient-specific devices described herein are expected to improve delivery into the patient's body, placement at the treatment site, and/or interaction with the patient's anatomy.

In embodiments where the patient-specific surgical plan includes a specific surgical procedure to implant a medical device, the treatment planning module 118 can also store various types of implant surgery information, such as implant parameters (e.g., types, dimensions), availability of implants, aspects of a pre-operative plan (e.g., initial implant configuration, detection and measurement of the patient's anatomy, etc.), FDA requirements for implants (e.g., specific implant parameters and/or characteristics for compliance with FDA regulations), or the like. In some embodiments, the treatment planning module 118 can convert the implant surgery information into formats useable for machine-learning based models and algorithms. For example, the implant surgery information can be tagged with particular identifiers for formulas or can be converted into numerical representations suitable for supplying to the trained machine learning model(s). The treatment planning module 118 can also store information regarding the patient's anatomy, such as two- or three-dimensional images or models of the anatomy, and/or information regarding the biology, geometry, and/or mechanical properties of the anatomy. The anatomy information can be used to inform implant design and/or placement.

The disease progression module 120 can be used to analyze, predict, and/or model disease progression for a particular patient. As described in detail below, the disease progression module 120 can estimate the rate of disease progression for the patient under a variety of different circumstances, including (a) if no surgical intervention occurs, and (b) if the surgical plan (e.g., surgical procedures identified by the treatment planning module 118) is performed. The disease progression module 120 can therefore include an algorithm, machine learning model, or other software analytical tool for predicting disease progression in a particular patient.

In some embodiments, the disease progression module 120 includes a machine learning model or other software module that can be trained based off a plurality of reference patient data sets that includes, in addition to the patient data described above, disease progression metrics for each of the reference patients. The progression metrics can include measurements for disease metrics over a period of time. Suitable metrics may include spinopelvic parameters (e.g., lumbar lordosis, pelvic tilt, sagittal vertical axis (SVA), cobb angel, coronal offset, etc.), disability scores, functional ability scores, flexibility scores, VAS pain scores, or the like. The progression of the metrics for each reference patient can be correlated to other patient information for the specific reference patient (e.g., age, sex, height, weight, activity level, diet, etc.). The disease metrics can include values over a period of time. For example, the reference patient data may include values of disease metrics on a daily, weekly, monthly, bi-monthly, yearly, or other basis. By measuring the metrics over a period of time, changes in the values of the metrics can be tracked as an estimate of disease progression and correlated to other patient data. Similarly, a need for and timing of additional surgical procedures can be predicted.

In some embodiments, the disease progression module 120 can therefore estimate the rate of disease progression for a particular patient. The progression may be estimated by providing estimated changes in one or more disease metrics over a period of time (e.g., X% increase in a disease metric per year). The rate can be constant (e.g., 5% increase in pelvic tilt per year) or variable (e.g., 5% increase in pelvic tilt for a first year, 10% increase in pelvic tilt for a second year, etc.). In some embodiments, the estimated rate of progression can be transmitted to a surgeon or other healthcare provider as part of a surgical plan, as described in greater detail below. The estimated rate of progression can also be utilized to determine timing for performing a first surgical procedure and/or a second surgical procedure, as also described in greater detail below.

As a non-limiting example, a particular patient who is a fifty-five-year-old male may have a SVA value of 6 mm. The disease progression module 120 can analyze patient reference data sets to identify disease progression for individual reference patients having one or more similarities with the particular patient (e.g., individual patients of the reference patients who have an SVA value of about 6 mm and are approximately the same age, weight, height, and/or sex of the patient). Based on this analysis, the disease progression module 120 can predict the rate of disease progression if no surgical intervention occurs (e.g., the patient's VAS pain scores may increase 5%, 10%, or 15% annually if no surgical intervention occurs, the SVA value may continue to increase by 5% annually if no surgical intervention occurs, etc.). The disease progression module 120 can also predict the rate of disease progression or the onset of additional pathologies if a first surgical procedure were to be performed (e.g., the patient's VAS pain scores may remain constant for 5 years post surgery, and then increase 5% annually thereafter unless a second surgical procedure is performed).

As described in greater detail below in Section B, the multi-stage surgical treatment plans and/or associated patient-specific implants described herein can therefore be at least partially based on the estimated rates of disease progression, enabling the modeling of different outcomes over a desired period of times. Additionally, the models/simulations can account for any number of additional diseases or conditions to predict the patient's overall health, mobility, or the like. These additional diseases or conditions can, in combination with other patient health factors (e.g., height, weight, age, activity level, etc.) be used to generate a patient health score reflecting the overall health of the patient. The patient health score can be displayed for surgeon review and/or incorporated into the estimation of disease progression. Accordingly, the present technology can generate one or more virtual simulations of the predicted disease progression to demonstrate how the patient's anatomy is predicted to change over time. Physician input can be used to generate or modify the virtual simulation(s). The present technology can generate one or more post-treatment virtual simulations based on the received physician input for review by the healthcare provider, patient, etc.

In some embodiments, the present technology can also predict, model, and/or simulate disease progression based on one or more potential surgical plans, including multi-stage surgical plans. For example, the disease progression module 120 may simulate what a patient's anatomy and/or spinal metrics may be 1, 2, 5, or 10 years post-surgery for several different surgical plans. The simulations may also incorporate non-surgical factors, such as patient age, height, weight, sex, activity level, other health conditions, or the like, as previously described. The system and/or a surgeon can use the disease progression to aid in selecting which surgical plan provides the best long-term efficacy, as described below. These simulations can also be used to determine patient-specific corrections that compensate for the projected diseases progression.

Accordingly, in some embodiments, multiple disease progression models (e.g., two, three, four, five, six, or more) are simulated to provide disease progression data for several different multi-stage surgical plans. For example, the disease progression module can generate models that predict post-surgical disease progression for each of three different multi-stage surgical plans. A surgeon or other healthcare provider can review the disease progression models and, based on the review, select which of the three multi-stage surgical plans is likely to provide the patient with the best long-term outcome.

Based off of the modeled disease progression, the systems and methods described herein can also (i) identify a recommended timing for surgical intervention, and/or (ii) identify a recommended type of surgical procedure for the patient. In some embodiments, the present technology therefore includes an intervention timing module 121 that includes an algorithm, machine learning model, or other software analytical tool for determining the optimal time for surgical intervention in a particular patient. This can be done, for example, by analyzing patient reference data that includes (i) pre-operative disease progression metrics for individual reference patients, (ii) disease metrics at the time of surgical intervention for individual reference patients, (iii) post-operative disease progression metrics for individual reference patients, and/or (iv) scored surgical outcomes for individual reference patients. The intervention timing module 121 can compare the disease metrics for a particular patient to the reference patient data sets to determine, for similar patients, the point of disease progression at which surgical intervention produced the most favorable outcomes. For multi-stage surgical plans that include a first surgical procedure and a second surgical procedure, the intervention timing module 121 can similarly identify the point of disease progression at which the first surgical procedure should be performed and the point of disease progression at which the second surgical procedure should be performed.

As a non-limiting example, the reference patient data sets may include data associated with reference patients' sagittal vertical axis. The data can include (i) sagittal vertical axis values for individual patients over a period of time before surgical intervention (e.g., how fast and to what degree the sagittal vertical axis value changed), (ii) sagittal vertical axis of the individual patients at the time of a first surgical procedure, (iii) the change in sagittal vertical axis after the first surgical procedure, (iv) the degree to which the first surgical procedure was successful (e.g., based on pain, quality of life, or other factors), (v) an amount of time between the first surgical procedure and a second surgical procedure, if needed, and (vi) the degree to which the second surgical procedure was successful. Based on the foregoing data, the intervention timing module 121 can, based on a particular patient's sagittal vertical axis value, identify at which point a first surgical procedure will have the highest likelihood of producing the most favorable outcome, as well as predict when a second surgical procedure will be necessary and/or may improve the patient's long-term outlook. Of course, the foregoing metric is provided by way of example only, and the intervention timing module 121 can incorporate other metrics (e.g., lumbar lordosis, pelvic tilt, sagittal vertical axis, cobb angel, coronal offset, disability scores, functional ability scores, flexibility scores, VAS pain scores) instead of or in combination with sagittal vertical axis to predict the time at which surgical interventions have the highest probability of providing a favorable outcome for the particular patient.

The intervention timing module 121 may also incorporate one or more mathematical rules based on value thresholds for various disease metrics. For example, the intervention timing module 121 may indicate surgical intervention is necessary if one or more disease metrics exceed a predetermined threshold or meet some other criteria. Representative thresholds that indicate surgical intervention may be necessary include SVA values greater than 7 mm, a mismatch between lumbar lordosis and pelvic incidence greater than 10 degrees, a cobb angle of greater than 10 degrees, and/or a combination of cobb angle and LL/PI mismatch greater than 20 degrees. Of course, other threshold values and metrics can be used; the foregoing are provided as examples only. In some embodiments, the foregoing rules can be tailored to specific patient populations (e.g., for males over 50 years of age, an SVA value greater than 7 mm indicates the need for surgical intervention). If a particular patient does not exceed the thresholds indicating surgical intervention is recommended, the intervention timing module 121 may provide an estimate for when the patient's metrics will exceed one or more thresholds, thereby providing the patient with an estimate of when surgical intervention may become recommended.

In some embodiments, the treatment planning module 118 identifies one or more types of surgical procedures for the patient based at least in part on the disease progression of the patient determined using the disease progression module 120 and/or the intervention timing module 121. The treatment planning module 118 may also incorporate one or more mathematical rules for identifying surgical procedures. As a non-limiting example, if a LL/PI mismatch is between 10 and 20 degrees, the treatment planning module 118 may recommend an anterior fusion surgery, but if the LL/PI mismatch is greater than 20 degrees, the treatment planning module may recommend both anterior and posterior fusion surgery. As another non-limiting example, if a SVA value is between 7 mm and 15 mm, the treatment planning module may recommend posterior fusion surgery, but if the SVA is above 15 mm, the treatment planning module may recommend both posterior fusion surgery and anterior fusion surgery. Of course, other rules can be used; the foregoing are provided as examples only.

Without being bound by theory, incorporating disease progression modeling into the patient-specific surgical plans described herein may even further increase the effectiveness of the procedures and/or provide a surgeon more data by which to evaluate various surgical plans. For example, in many cases it may be disadvantageous to operate after a patient's disease progresses to an irreversible or unstable state. However, it may also be disadvantageous to operate too early, such as before the patient's disease is causing symptoms and/or if the patient's disease may not progress further. The disease progression module 120 and/or the intervention timing module 121 can therefore help identify the window of time during which surgical intervention in a particular patient has the highest probability of providing a favorable outcome for the patient. The disease progression module 120 and/or the intervention timing module 121 can also help with predicting when a second surgical intervention may be needed if a first surgical intervention were to be performed. In this way, the disease progression module 120 and/or the intervention timing module 121 can provide a holistic overview of likely outcomes if a given surgical plan were to be implemented.

The surgical plan(s) generated by the treatment planning module 118 can be transmitted via the communication network 104 to the client computing device 102 for output to a user (e.g., clinician, surgeon, healthcare provider, patient). In some embodiments, the client computing device 102 includes or is operably coupled to a display 122 for outputting the treatment plan(s). The display 122 can include a graphical user interface (GUI) for visually depicting various aspects of the surgical plan(s). For example, the display 122 can show various aspects of a surgical procedure to be performed on the patient, such as the surgical approach, treatment levels, corrective maneuvers, tissue resection, and/or implant placement. To facilitate visualization, the surgical plan can include a virtual model of the surgical procedure that can be displayed via the display 122. The display 122 may also display additional aspects of the surgical plan, such as a first surgical procedure, predicted post-operative patient metrics following the first surgical procedure, predicted disease progression metrics following the first surgical procedure, a second surgical procedure, predicted post-operative patient metrics following a second surgical procedure, predicted disease progression metrics following the second surgical procedure, etc. As another example, the display 122 can show a design for a medical device to be implanted in the patient in accordance with the transmitted surgical plan, such as a two- or three-dimensional model of the device design. The display 122 can also show patient information, such as two- or three-dimensional images or models of the patient's anatomy where the surgical procedure is to be performed and/or where the device is to be implanted.

In some embodiments, one or more aspects of the surgical plan are displayed using the surgical plan review program 125. For example, the review program 125, which may be implemented as a mobile phone application, a computer application, or the like, can display (e.g., via the display 122) one or more aspects of the surgical plan (e.g., a first surgical procedure, a second surgical procedure, a virtual model of patient anatomy, an implant, etc.). The review program 125 may provide an interactive interface that further enables a surgeon to select between different patients, select between different surgical plans for the same patient, compare surgical plans for the same patient, review the status of a surgical plan, provide feedback on a proposed surgical plan, accept a surgical plan, reject a surgical plan, etc. The review program 125 may further enable a surgeon or other user to select between different views of a virtual model of patient anatomy, and/or different views of a patient-specific implant to be used in the surgical plan.

In some embodiments, the medical device design(s) generated by the treatment planning module 118 can be transmitted from the client computing device 102 and/or server 106 to a manufacturing system 124 for manufacturing a corresponding medical device. The manufacturing system 124 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site.

Various types of manufacturing systems are suitable for use in accordance with the embodiments herein. For example, the manufacturing system 124 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 124 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 124 can manufacture one or more patient-specific medical devices based on fabrication instructions or data (e.g., CAD data, 3D data, digital blueprints, stereolithography data, or other data suitable for the various manufacturing technologies described herein). Different components of the system 100 can generate at least a portion of the manufacturing data used by the manufacturing system 124. The manufacturing data can include, without limitation, fabrication instructions (e.g., programs executable by additive manufacturing equipment, subtractive manufacturing equipment, etc.), 3D data, CAD data (e.g., CAD files), CAM data (e.g., CAM files), path data (e.g., print head paths, tool paths, etc.), material data, tolerance data, surface finish data (e.g., surface roughness data), regulatory data (e.g., FDA requirements, reimbursement data, etc.), or the like. The manufacturing system 124 can analyze the manufacturability of the implant design based on the received manufacturing data. The implant design can be finalized by altering geometries, surfaces, etc. and then generating manufacturing instructions. In some embodiments, the server 106 generates at least a portion of the manufacturing data, which is transmitted to the manufacturing system 124.

The manufacturing system 124 can generate CAM data, print data (e.g., powder bed print data, thermoplastic print data, photo resin data, etc.), or the like and can include additive manufacturing equipment, subtractive manufacturing equipment, thermal processing equipment, or the like. The additive manufacturing equipment can be 3D printers, stereolithography devices, digital light processing devices, fused deposition modeling devices, selective laser sintering devices, selective laser melting devices, electronic beam melting devices, laminated object manufacturing devices, powder bed printers, thermoplastic printers, direct material deposition devices, or inkjet photo resin printers, or like technologies. The subtractive manufacturing equipment can be CNC machines, electrical discharge machines, grinders, laser cutters, water jet machines, manual machines (e.g., milling machines, lathes, etc.), or like technologies. Both additive and subtractive techniques can be used to produce implants with complex geometries, surface finishes, material properties, etc. The generated fabrication instructions can be configured to cause the manufacturing system 124 to manufacture the patient-specific orthopedic implant that matches or is therapeutically the same as the patient-specific design. In some embodiments, the patient-specific medical device can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The surgical plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 118 generates control instructions configured to cause a surgical robot (e.g., robotic surgery systems, navigation systems, etc.) to partially or fully perform a surgical procedure. The control instructions can be transmitted to the robotic apparatus by the client computing device 102 and/or the server 106.

Following the treatment of the patient in accordance with the surgical plan, treatment progress can be monitored over one or more time periods to update the data analysis module 116, treatment planning module 118, disease progression module 120, and/or intervention timing module 121. Post-treatment data can be added to the reference data stored in the database 110. The post-treatment data can be used to train machine learning models for developing patient-specific treatment plans, patient-specific medical devices, or combinations thereof.

It shall be appreciated that the components of the system 100 can be configured in many different ways. For example, in alternative embodiments, the database 110, the data analysis module 116, the treatment planning module 118, the disease progression module 120, and/or the intervention timing module 121 can be components of the client computing device 102, rather than the server 106. As another example, the database 110, the data analysis module 116, the treatment planning module 118, the disease progression module 120, and/or the intervention timing module 121 can be located across a plurality of different servers, computing systems, or other types of cloud-computing resources, rather than at a single server 106 or client computing device 102.

Additionally, in some embodiments, the system 100 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like.

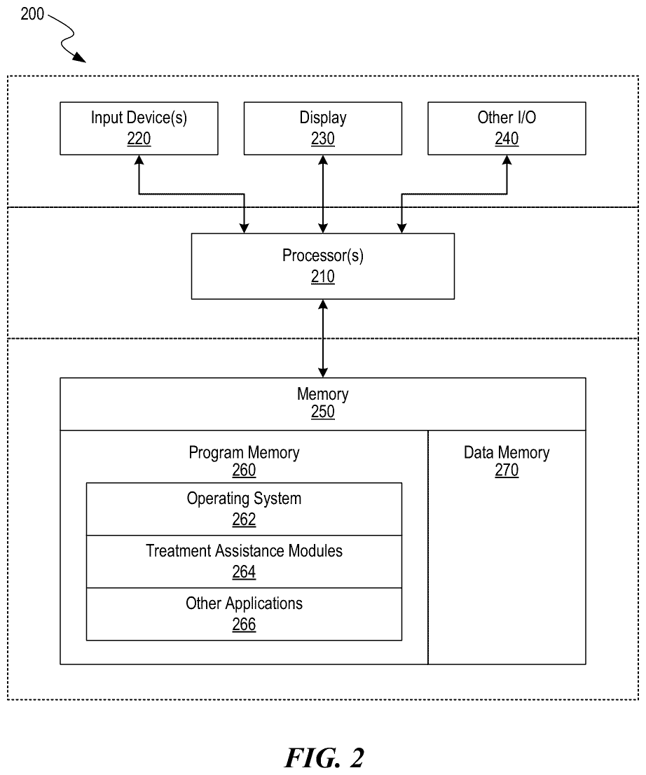

FIG. 2 illustrates a computing device 200 suitable for use in connection with the system 100 of FIG. 1, according to an embodiment. The computing device 200 can be incorporated in various components of the system 100 of FIG. 1, such as the client computing device 102 or the server 106. The computing device 200 includes one or more processors 210 (e.g., CPU(s), GPU(s), HPU(s), etc.). The processor(s) 210 can be a single processing unit or multiple processing units in a device or distributed across multiple devices. The processor(s) 210 can be coupled to other hardware devices, for example, with the use of a bus, such as a PCI bus or SCSI bus. The processor(s) 210 can be configured to execute one more computer-readable program instructions, such as program instructions to carry out of any of the methods described herein.

The computing device 200 can include one or more input devices 220 that provide input to the processor(s) 210, e.g., to notify it of actions from a user of the device 200. The actions can be mediated by a hardware controller that interprets the signals received from the input device and communicates the information to the processor(s) 210 using a communication protocol. Input device(s) 220 can include, for example, a mouse, a keyboard, a touchscreen, an infrared sensor, a touchpad, a wearable input device, a camera- or image-based input device, a microphone, or other user input devices.

The computing device 200 can include a display 230 used to display various types of output, such as text, models, virtual procedures, surgical plans, implants, graphics, and/or images (e.g., images with voxels indicating radiodensity units or Hounsfield units representing the density of the tissue at a location). In some embodiments, the display 230 provides graphical and textual visual feedback to a user. The processor(s) 210 can communicate with the display 230 via a hardware controller for devices. In some embodiments, the display 230 includes the input device(s) 220 as part of the display 230, such as when the input device(s) 220 include a touchscreen or is equipped with an eye direction monitoring system. In alternative embodiments, the display 230 is separate from the input device(s) 220. Examples of display devices include an LCD display screen, an LED display screen, a projected, holographic, or augmented reality display (e.g., a heads-up display device or a head-mounted device), and so on.

Optionally, other I/O devices 240 can also be coupled to the processor(s) 210, such as a network card, video card, audio card, USB, firewire or other external device, camera, printer, speakers, CD-ROM drive, DVD drive, disk drive, or Blu-Ray device. Other I/O devices 240 can also include input ports for information from directly connected medical equipment such as imaging apparatuses, including MRI machines, X-Ray machines, CT machines, etc. Other I/O devices 240 can further include input ports for receiving data from these types of machine from other sources, such as across a network or from previously captured data, for example, stored in a database.

In some embodiments, the computing device 200 also includes a communication device (not shown) capable of communicating wirelessly or wire-based with a network node. The communication device can communicate with another device or a server through a network using, for example, TCP/IP protocols. The computing device 200 can utilize the communication device to distribute operations across multiple network devices, including imaging equipment, manufacturing equipment, etc.

The computing device 200 can include memory 250, which can be in a single device or distributed across multiple devices. Memory 250 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 250 is a non-transitory computer-readable storage medium that stores, for example, programs, software, data, or the like. In some embodiments, memory 250 can include program memory 260 that stores programs and software, such as an operating system 262, one or more treatment assistance modules 264, and other application programs 266. The treatment assistance module(s) 264 can include one or more modules configured to perform the various methods described herein (e.g., the data analysis module 116 and/or treatment planning module 118 described with respect to FIG. 1). Memory 250 can also include data memory 270 that can include, e.g., reference data, configuration data, settings, user options or preferences, etc., which can be provided to the program memory 260 or any other element of the computing device 200.

### B. Select Methods of Modeling and Designing Multi-Stage Surgical Plans

The present technology includes systems and methods for generating patient-specific surgical plans, including multi-stage patient-specific surgical plans, and transmitting the patient-specific surgical plans to a surgeon or other healthcare provider for review, feedback, modification, and/or approval. As described below, the multi-stage patient-specific surgical plans can include, among other things, surgical plans having two or more discrete treatment stages, with each treatment stage having a particular surgical or other medical intervention.

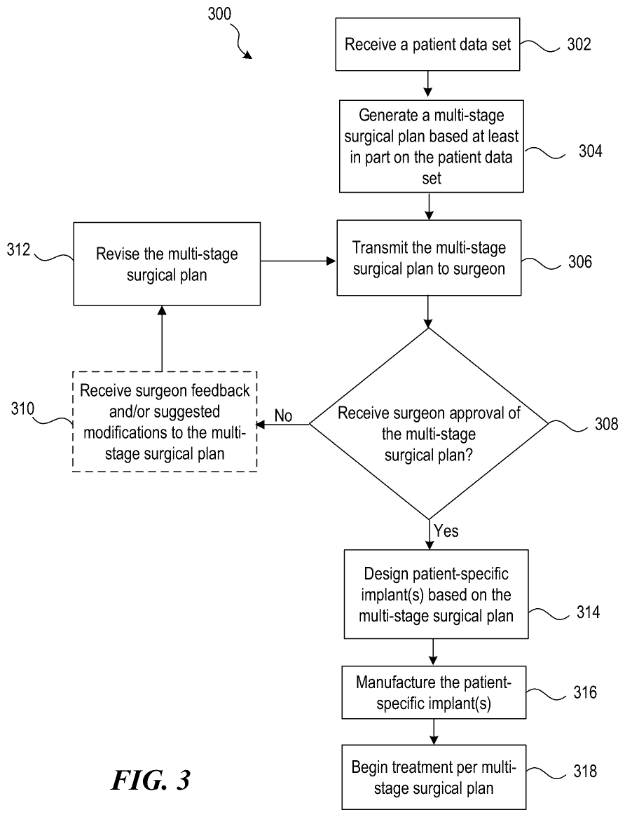

FIG. 3 is a flow diagram illustrating a method 300 for providing patient-specific medical care, according to an embodiment of the present technology. Some or all of the method 300 can be performed by various computing systems or software modules, including, for example, the computing systems described above with respect to FIGS. 1 and 2. The method 300 can begin at block 302 by receiving a patient data set for a particular patient in need of medical treatment. The patient data set can include data representative of the patient's condition, anatomy, pathology, symptoms, medical history, preferences, and/or any other information or parameters relevant to the patient. For example, the patient data set can include surgical intervention data, treatment outcome data, progress data (e.g., surgeon notes), patient feedback (e.g., feedback acquired using quality of life questionnaires, surveys), clinical data, patient information (e.g., demographics, sex, age, height, weight, type of pathology, occupation, activity level, tissue information, health rating, comorbidities, health related quality of life (HRQL)), vital signs, diagnostic results, medication information, allergies, diagnostic equipment information (e.g., manufacturer, model number, specifications, user-selected settings/configurations, etc.) or the like. The patient data set can also include image data, such as camera images, Magnetic Resonance Imaging (MRI) images, ultrasound images, Computerized Aided Tomography (CAT) scan images, Positron Emission Tomography (PET) images, X-Ray images, and the like. In some embodiments, the patient data set includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine. The patient data set can be received at a server, computing device, or other computing system. For example, in some embodiments the patient data set can be received by the server 106 shown in FIG. 1. In some embodiments, the computing system that receives the patient data set at block 302 also stores one or more software modules (e.g., the data analysis module 116, the treatment planning module 118, the disease progression module 120, and/or the intervention timing module 121, shown in FIG. 1, or additional software modules for performing various operations of the method 300).

In some embodiments, the received patient data set can include disease metrics such as lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine). In some embodiments, the disease metrics are not included in the patient data set, and the method 300 includes determining (e.g., automatically determining) one or more of the disease metrics based on the patient image data, as described below. In some embodiments, the received patient data can include functional mobility test scores (e.g., step test, six-meter walk test, sit-to-stand test, timed up and go test, etc.). The received patient data set can include additional subjective test scores that reflect aspects of the patient condition, such as pain tests (e.g., Visual Analog Scale (VAS) pain scores, Low Back Pain Rating scale scores, etc.), disability tests (e.g., Oswestry Disability Index scores, Quebec back pain disability test scores, etc.), quality of life tests (e.g., Quality of Life Scale scores), etc.

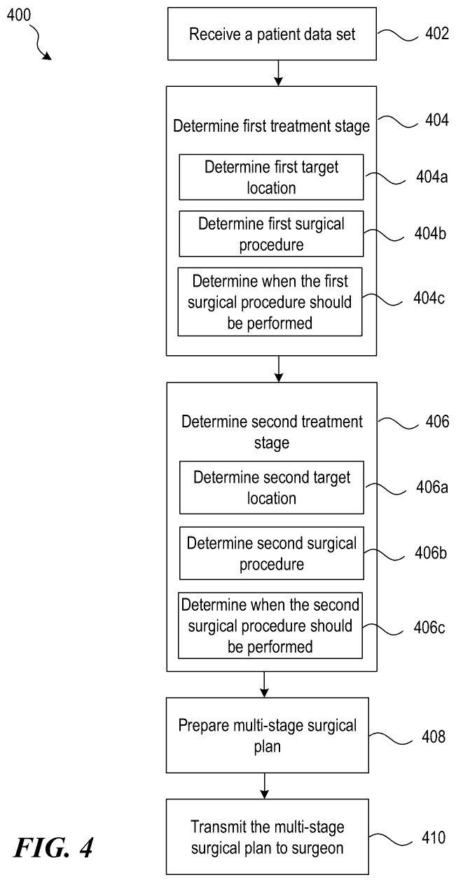

The method 300 can continue at block 304 by generating a multi-stage surgical plan based at least in part on the patient data set received at block 302. As described in detail below, the multi-stage surgical plan can include two or more treatment stages. Each treatment stage can include a target location or region of interest for performing a surgical procedure, and one or more specific procedures or interventions to be performed at the region of interest. The surgical plan can also include predicted post-operative data associated with performing the first and second treatment stages. For example, the surgical plan may include a predicted or target post-operative anatomical configuration shown as a two- or three-dimensional virtual model for both the first treatment stage and the second treatment stage. In some embodiments, the surgical plan also includes additional predicted post-operative analytics, such as predicted disease progression following the first and/or second treatment stages, predicted patient satisfaction following the first and/or second treatment stages, predicted patient mobility following the first and/or second treatment stages, predicted patient pain following the first and/or second treatment stages, predicted patient quality of life following the first and/or second treatment stages, etc. In some embodiments, generating the multi-stage surgical plan at block 304 includes performing some or all of the operations described below with reference to FIG. 4.

Once the multi-stage surgical plan is generated at block 304, the method 300 can continue at block 306 by transmitting the multi-stage surgical plan to a surgeon. In some embodiments, the same computing system used at blocks 302 and 304 can transmit the surgical plan to a computing device for surgeon review (e.g., the client computing device 102 described in FIG. 1). This can include directly transmitting the multi-stage surgical plan to the computing device or uploading the first and second surgical plans to a cloud or other storage system for subsequent downloading. The multi-stage surgical plan can be digitally displayed as a surgical report on one or more display screens for ease of review, editing, annotation, the like. In some embodiments, the surgical plan can therefore be stored as computer-executable instructions that can be executed via the surgical plan review module 123 on the client computing device 102 of FIG. 1. As a result, the surgical plan can be reviewed using the surgical plan review program 125, shown in FIG. 1.

The surgeon can review the multi-stage surgical plan and, at block 308, approve or disapprove of the surgical plan. For example, the surgeon may review the multi-stage surgical plan using the surgical plan review program 125 (FIG. 1) to determine whether the surgeon deems the surgical plan acceptable. This may include, for example, reviewing the surgical plans target locations, surgical procedures, target/predicted post-operative anatomical configurations, and predicted post-operative patient metrics.

In some embodiments, the surgeon may not approve the multi-stage surgical plan at block 308. In such embodiments, the surgeon can optionally provide feedback and/or suggested modifications to the surgical plan (e.g., by adjusting the virtual model or changing one or more aspects about the plan, providing comments on or more requested changes to the surgical plan, etc.). Accordingly, the method 300 can optionally include receiving (e.g., via the computing system) the surgeon feedback and/or suggested modifications at block 310. This may include, for example, modifying target locations for surgical intervention, surgical procedures, dates of surgery, sequence of surgical procedures, and/or target post-operative anatomical configurations. If surgeon feedback and/or suggested modifications are received at block 310, the method 300 can continue at block 312 by revising (e.g., automatically revising via the computing system) the multi-stage surgical plan based at least in part on the surgeon feedback and/or suggested modifications received at block 310. In some embodiments, the surgeon does not provide feedback and/or suggested modifications if they reject the surgical plan. In such embodiments, block 310 can be omitted, and the method 300 can continue at block 312 by revising (e.g., automatically revising via the computing system) the multi-stage surgical plans by selecting new and/or additional reference patient data sets and/or generating a new candidate multi-stage surgical plan. The revised and/or new multi-stage surgical plan can then be transmitted to the surgeon for review. The operations at blocks 306, 308, 310, and 312 can be repeated as many times as necessary until the surgeon selects and approves a particular surgical plan.

Once surgeon approval of a surgical plan is received at block 308, the method 300 can continue at block 314 by designing (e.g., via the same computing system that performed blocks 302-308) one or more patient-specific implants based on the selected surgical plan. In some embodiments, the first surgical procedure is associated with one or more first patient-specific implants, and the second surgical procedure is associated with one or more second patient-specific implants. The patient-specific implants can be designed based on the target locations and surgical procedures included in the selected surgical plan. The patient-specific implants can also be specifically designed such that, when implanted in the particular patient at the target location using the identified surgical procedure, they direct the patient's anatomy to occupy the target post-operative anatomical configuration associated with the first surgical procedure and/or the second surgical procedure (e.g., transforming the patient's anatomy from the patient's native anatomical configuration to the corrected anatomical configuration). The patient-specific implant can be designed such that, when implanted, it causes the patient's anatomy to occupy the corrected anatomical configuration for the expected service life of the implant (e.g., 5 years or more, 10 years or more, 20 years or more, 50 years or more, etc.). In some embodiments, the patient-specific implant is designed solely based on the virtual model of the corrected anatomical configuration and/or without reference to pre-operative patient images.

The patient-specific implant can be any of the implants described herein or in any patent references incorporated by reference herein. For example, the patient-specific implant can include one or more of screws (e.g., bone screws, spinal screws, pedicle screws, facet screws), interbody implant devices (e.g., intervertebral implants), cages, plates, rods, discs, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.). Examples of patient-specific implants that can be designed at block 314 are described in U.S. application Ser. Nos. 16/048,167, 16/242,877, 16/207,116, 16/352,699, 16/383,215, 16/569,494, 16/699,447, 16/735,222, 16/987,113, 16/990,810, 17/085,564, 17/100,396, 17/342,329, 17/518,524, 17/531,417, 17/835,777, 17/851,487, 17/867,621, and 17/842,242, each of which is incorporated by reference herein in its entirety.

In some embodiments, designing the implant at block 314 can optionally include generating fabrication instructions for manufacturing the implant. For example, the computing system may generate computer-executable fabrication instructions that, when executed by a manufacturing system, cause the manufacturing system to manufacture the implant. In embodiments in which the multi-stage surgical plan calls for one or more first implants and one or more second implants, fabrication instructions can optionally be generated for both the one or more first implants and the one or more second implants simultaneously. Alternatively, fabrication instructions can be generated for the one or more first implants to be used with the first surgical procedure, and a design for the one or more second implants associated with the second surgical procedure can be stored for use at a later date (e.g., closer to the date of the second surgical procedure) to generate fabrication instructions for the one or more second implants.

In some embodiments, the patient-specific implants are designed at block 314 only after the surgeon has selected a surgical plan. Accordingly, in some embodiments, the implant designs are neither transmitted to the surgeon with the surgical plan at block 308, nor manufactured before receiving surgeon approval of the surgical plan. Without being bound by theory, waiting to design the patient-specific implants until after the surgeon approves the surgical plan may increase the efficiency of the method 300 and/or reduce the resources necessary to perform the method 300. In other embodiments, one or more patient-specific implants can be designed and included in the surgical plans transmitted to the surgeon at block 306. Accordingly, in some embodiments the operation at block 314 can be included within the block 304.

The method 300 can continue at block 316 by manufacturing the patient-specific implants. In embodiments in which the multi-stage surgical plan includes one or more first implants and one or more second implants, both the one or more first implants and the one or more second implants can optionally be manufactured substantially simultaneously. Alternatively, it may be advantageous to initially only manufacture the one or more first implants associated with the first surgical procedure. The one or more second implants can be manufactured at a later date as the date for the second surgical procedure approaches.

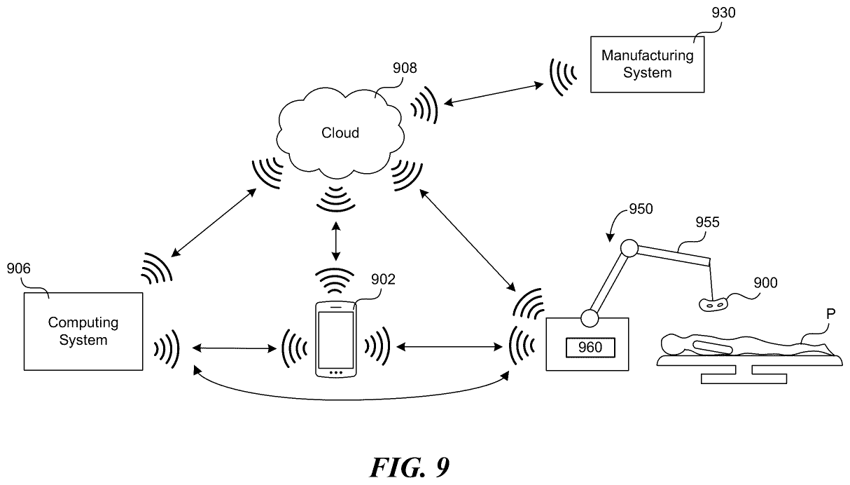

Regardless of whether the operation at block 316 includes manufacturing the first implants or the second implants, the implants can be manufactured using additive manufacturing techniques, such as 3D printing, stereolithography, digital light processing, fused deposition modeling, selective laser sintering, selective laser melting, electronic beam melting, laminated object manufacturing, powder bed printing, thermoplastic printing, direct material deposition, or inkjet photo resin printing, or like technologies, or combination thereof. Alternatively or additionally, the implants can be manufactured using subtractive manufacturing techniques, such as CNC machining, electrical discharge machining (EDM), grinding, laser cutting, water jet machining, manual machining (e.g., milling, lathe/turning), or like technologies, or combinations thereof. The implants may be manufactured by any suitable manufacturing system (e.g., the manufacturing system 124 shown in FIG. 1 or the manufacturing system 930 described below with respect to FIG. 9). In some embodiments, the implants are manufactured by the manufacturing system executing the computer-readable fabrication instructions generated by the computing system at block 316.

Once the implant is manufactured at block 316, the method 300 can continue at block 318 by beginning treatment per the multi-stage surgical plan. This may include, for example, preparing for and performing the first surgical procedure. Aspects of the surgical plan, such as some or all of the first and second surgical procedures, can be performed manually, by a robotic surgical platform (e.g., a surgical robot), or a combination thereof. In embodiments in which the surgical procedure is performed at least in part by a robotic surgical platform, the surgical plan can include computer-readable control instructions configured to cause the surgical robot to perform, at least partly, the patient-specific surgical procedure.

The method 300 can be implemented and performed in various ways. In some embodiments, the operations at blocks 302-314 can be performed by a computing system associated with a first entity, block 316 can be performed by a manufacturing system associated with a second entity, and block 318 can be performed by a surgical provider, surgeon, and/or robotic surgical platform associated with a third entity. Any of the foregoing blocks may also be implemented as computer-readable instructions stored in memory and executable by one or more processors of the associated computing system(s).

FIG. 4 is a flowchart of a method 400 for generating a multi-stage surgical plan in accordance with embodiments of the present technology. Some or all of the method 400 can be performed by various computing systems or software modules, including, for example, the computing systems described above with respect to FIGS. 1 and 2. The method 400 can begin at block 402 by receiving a patient data set. The operation performed at block 402 can be the same as, or at least generally similar to, the operation performed at block 302 of the method 300 described with reference to FIG. 3. Accordingly, the patient data set can include any of the information described above with reference to the block 302 of the method 300.

The method 400 can continue at block 404 by determining a first treatment stage for the patient. The operation of determining the first treatment stage can include, several suboperations. For example, the operation of determining the first treatment stage at block 404 can include, at block 404a, determining a first target location to be involved in the first surgical stage. For example, in the context of spinal surgery, one or more vertebral levels may be identified for surgical intervention. In some embodiments, the vertebral level is a cervical vertebral level (e.g., C1-C5), a thoracic vertebral level (e.g., T1-T12), a lumbar vertebral level (e.g., L1-L5), and/or the sacrum. In some embodiments, the first target location includes a specific range of vertebral levels to be involved in a surgery (e.g., L1-L3, L3-L5, L4-T12, C1-C3, etc.). The first target location may include two, three, four, five, or more vertebral levels. Of course, the foregoing target locations are provided by way of example only, and the present technology is not limited to the anatomical locations listed above. Indeed, in some embodiments the first target location may include anatomical structures other than the spine, such as the hip, knee, ankle, shoulder, elbow, wrist, hand, the jaw, the skull, or other anatomical locations, as described throughout this Detailed Description.

The first target location can be determined by reviewing image data of the patient. In some embodiments, a computing system (e.g., the server 106 of FIG. 1) and/or one or more software modules (e.g., the treatment planning module 118 of FIG. 1) can review and analyze patient image data and automatically identify the first target location. In such embodiments, a trained machine learning program or other software-based program can analyze patient image data, identify anatomical features, extract measurements from the patient image data, compare the extracted measurements to reference data (e.g., predetermined thresholds or ranges associated with “healthy” patients normalized for age, sex, gender, etc.), and/or identify anatomical regions that are candidates for surgical correction. Alternatively or additionally, the first target location can be identified and/or confirmed through other suitable means, such as via a technician or healthcare provider reviewing image data and identifying anatomical deformities.

The operation of determining the first treatment stage at block 404 can also include, at block 404b, determining a first surgical procedure to be performed. The first surgical procedure can be associated with the first target location. In the context of spinal surgery, representative surgical procedures include spinal fusion, artificial disc replacement, vertebroplasty, kyphoplasty, spinal laminectomy/decompression, discectomy, facetectomy, foraminotomy, or other spine surgery procedures. Examples of spinal fusion surgery include posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). Accordingly, in some embodiments, determining the first surgical procedure includes determining a particular approach to the spine (e.g., anterior, lateral, posterior, etc.). The foregoing are provided by way of example only, and the present technology can include identifying any type of spinal or other surgical procedures at block 304b.

The first surgical procedure can be determined using any of the methods and systems described herein. For example, in some embodiments the server 106 of FIG. 1 and/or the associated software modules (e.g., the treatment planning module 118) can identify one or more surgical procedures based on, for example, user input, the received or extracted patient data, and/or the identified target location(s). For example, if the server 106 determines that a patient is suffering from disc degeneration from L3-L5, the system may recommend a PLIF procedure to fuse L2-T12. Alternatively, the system may recommend an artificial disc replacement at L3-L4 and L4-L5 to correct the degeneration while preserving motion. The first surgical procedure can be identified using other methods and systems as well.

In some embodiments, the operation at block 404b can include reviewing and/or analyzing multiple types of surgical procedures and/or surgical steps to identify the first surgical procedure. In some embodiments, types of surgical procedures and/or surgical steps can be selected for inclusion (or eliminated from inclusion) based on, for example, user input, insurance coverage of the procedure or step, healthcare provider parameters (e.g., based on healthcare provider ranking/scores such as hospital/physician expertise, number of similar procedures performed, hospital ranking for procedure, etc.), healthcare resource parameters (e.g., diagnostic equipment, facilities, surgical equipment such as surgical robots), and/or other non-patient related information (e.g., information that can be used to score, predict outcomes and risk profiles for procedures for the present healthcare provider, and/or rank procedures).

The operation of determining the first treatment stage at block 404 can also include, at block 404c, determining when the first surgical procedure identified at block 404b should be performed. In some embodiments, this may include simulating or estimating disease progression for the patient (e.g., using the disease progression module 120 of the system 100, described with reference to FIG. 1) and identifying at what stage of progression is the first surgical procedure likely to have the most positive outcome. This may include, for example, comparing particular patient data to reference patient data to determine at which disease stage similarly situated patients had the most favorable surgical outcomes.

In some embodiments, determining when the first surgical procedure should be performed includes identifying a recommended date or range of dates to perform the first surgical procedure in order to obtain the most positive outcome. This may be done by reviewing patient data and comparing patient data to reference patient data, as previously described. For example, some patients with a chronic, degenerative condition may benefit from immediate surgical intervention to avoid further disease progression. In such embodiments, the timing of the first surgical procedure can be set as substantially immediate (e.g., within 30 days, within 60 days, within 90 days, etc.). On the other hand, some patients with acute injury (e.g., spinal cord injury) may benefit from delayed surgical intervention, e.g., to permit inflammation to reduce before performing surgery. In such embodiments, the timing of the first surgical procedure can be set within a time range (e.g., between 3 months and 12 months, between 3 months and 6 months) or at a future time (e.g., 3 months, 4 months, 5 months, 6 months, etc.). The first treatment stage can therefore have a fixed timing based on passage of time.

Alternatively, rather than specifying a specific time for the surgery, determining when the first surgical procedure should be performed can include identifying that one or more outcome-dependent health metrics should be changed before beginning the first treatment stage. This may include, for example, identifying one or more threshold health metrics. In such embodiments, patient metrics can be tracked over time (e.g., after approval of the multi-stage surgical plan), and, once the threshold metrics are met, the first surgical procedure can be performed. For example, in a patient with degenerative disc disease, a threshold metric may be disc height at a particular vertebral level (e.g., a disc height of 10 mm at L3-L4, 9 mm at L4-L5, 8 mm at L5-S1, etc.). In such embodiments, once the patient's disc height falls below the threshold (e.g., once the patient's L3-L4 disc height is less than 10 mm), the patient can be indicated as ready for the first surgical procedure. As another example, in a patient with an acute spinal injury with swelling, a threshold metric may be amount of swelling (e.g., swelling reduction of 60%, 70%, 80%, or 90%relative to current state). Once the patient's swelling reduction meets the threshold, the patient can be indicated as ready for the first surgical procedure. As another example, in a morbidly obese patient, a threshold metric may be a target weight or body mass index (“BMI”), with the first treatment stage commencing only after the patient's weight and/or BMI falls below the threshold metric. The first treatment stage can therefore have adaptive timing based on changes in patient pathology and/or health. As yet another example, a threshold metric can include a disability score (e.g., VAS, ODI, SRS22, etc.), or any other quantitative or qualitative metrics identified in this Detailed Description.

To determine the threshold metrics, a computing system can simulate likely outcomes if the patient were to undergo the first treatment stage at their current pathology/condition, at an improved pathology/health, and/or at a worsened pathology/health. For example, if a patient is morbidly obese, the computing system can simulate likely outcomes if the patient were to undergo the first treatment stage at the patient's current weight, at 90% of the patient's current weight, at 80% of the patient's current weight, at 110% of the patient's current weight, etc. Each of the simulations can provide predicted outcomes and probabilities of success. The outcomes can be scored verses the likelihood of the corresponding threshold being met (e.g., the outcomes can be weighted with the understanding that the more weight the patient must lose, the less likely the patient will meet the threshold). The scores can then be ranked to determine a suitable threshold metric value that is balanced between being achievable while also providing a better probability of achieving a favorable outcome. In some embodiments, multiple simulations at different patient metrics (e.g., patient weights) can be shared with a patient and/or patient healthcare provider to show how a change in a patient's overall health may impact the patient's prognosis. For example, the patient can be given predicted improvements in spine pathology associated with different amounts of reduction in weight or BMI to encourage the patient to lose weight.

In some embodiments, the timing of the first surgical procedure can include a combination of fixed timing (e.g., based on passage of time) and an adaptive timing (e.g., based on changes in patient pathology or condition). For example, the operation at block 404c may prescribe that the first surgical procedure should be performed once either (a) a threshold metric is met, or (b) a certain passage of time occurs. That is, the first surgical procedure can be prescribed to be performed following whichever of (a) or (b) occurs first. As another example, the operation at block 404c may prescribe that the first surgical procedure should be performed only after both (a) a threshold metric is met, and (b) a certain passage of time occurs.

There can also be additional operations associated with determining the first treatment stage. For example, in some embodiments the operation of determining the first treatment stage includes identifying or designing a first corrected anatomical configuration for the patient (the corrected anatomical configuration can also be referred to herein as the “planned configuration,” “optimized geometry,” “post-operative anatomical configuration,” or “target outcome”). The first corrected anatomical configuration can reflect the desired and/or predicted anatomy of the patient if the first treatment stage were to be performed. In some embodiments, one or more virtual models (two-dimensional models, three-dimensional models, etc.) can be generated that show the first corrected anatomical configuration. The virtual model may include some or all of the patient's anatomy within the first target location (e.g., any combination of tissue types including, but not limited to, bony structures, cartilage, soft tissue, vascular tissue, nervous tissue, etc.). In some embodiments, the first corrected anatomical configuration is identified/determined before the surgical procedure and/or target location. That is, a computing system or user can model a preferred anatomical outcome, and, based on the desired anatomical outcome, identify the first surgical procedure and the first target location that will achieve the desired anatomical outcome once performed.

In some embodiments, patient metrics associated with the first corrected anatomical configuration can also be calculated. In the context of spinal surgery, patient metrics may include, for example, coronal parameters, sagittal parameters, pelvic parameters, Cobb angles, shoulder tilt, iliolumbar angles, coronal balance, lordosis angles, intervertebral space height, or other similar spinal parameters. Similar as described above, the patient metrics can be determined before identifying the first surgical procedure and/or the first target location. That is, a computing system or user can use the patient metrics to identify a surgical procedure and target location that will achieve the patient metrics once performed.

Once the first treatment stage is determined, the method 400 can continue at block 406 by determining a second treatment stage for the patient. Similar to determining the first treatment stage, determining the second treatment stage can include several sub operations. For example, determining the second treatment stage can include, at block 406a, determining a second target location to be involved in the second surgical stage. The second target location can include any of the anatomical targets identified above with reference to the first target location, and can be determined using any of the techniques described above with reference to the first target location.

The second target location may be the same as or different than the first target location that is involved in the first treatment stage. For example, in some embodiments, the second target location may include one or more vertebral levels that are continuous or overlapping with one or more vertebral levels identified as the first target location. As a particular example, if the first target location included L4-S1, the second target location may include L2-L4. Alternatively, the second target location may be spaced apart from the first target location, e.g., by one or more vertebral segments. For example, if the first target location included L4-S1, the second target location may include T12-L2.

The operation of determining the second treatment stage at block 406 can also include, at block 406b, determining a second surgical procedure to be performed. The second surgical procedure can include any of the surgical procedures identified above with reference to the first surgical procedure, and can be identified using any of the techniques described above with reference to the first surgical procedure. In some embodiments, the second surgical procedure can be the same “type” of procedure as the first surgical procedure. For example, if the first surgical procedure was a spinal fusion procedure, the second surgical procedure may also be a spinal fusion procedure, just at a different target location, using one or more different device(s) (e.g., implant types, such as interbody cages vs. fixation plates or rods), using one or more different surgical approaches (e.g., lateral vs. anterior vs. posterior), or procedures. Accordingly, the second surgical procedure may have a different goal than the first surgical procedure, even if it is the same “type” of procedure. Continuing with the specific example from above, if the first target location was L4-S1 and the first surgical procedure was a fusion at L4-S1, the second surgical procedure may be a fusion procedure at the second target location of L2-L4 or T12-L2. Moreover, if the first surgical procedure was performed using an anterior approach, the second surgical procedure may be performed using a lateral or posterior approach.

In other embodiments, the second surgical procedure is a different “type” of procedure than the first surgical procedure. As an example, the first surgical procedure may be a resection (e.g., bony resection) to remove abnormal or pathological tissue growth, and the second surgical procedure may be a fusion or other spinal surgery at the same level as the first surgical procedure to provide stability and/or to reduce patient pain. Of course, the foregoing are provided by way of example only, and the first and second surgical procedures can include any of the procedures identified throughout this Detailed Description.

The operation of determining the second treatment stage at block 406 can also include, at block 406c, determining when the second surgical procedure should be performed. Similar to the operation described at block 404c for determining when the first surgical procedure should be performed, determining when the second surgical procedure should be performed can be based off of simulated disease progression and timing intervention for reference patients that had favorable outcomes. In such embodiments, the simulated progression can account for the first treatment stage being performed (e.g., to determine progression assuming the first surgical procedure at the first target location were performed). That is, the estimated disease progression can incorporate treatment received during the first treatment stage, even though such treatment has not yet occurred in the patient. The simulated progression may also account for likely changes in a patient's overall health (e.g., weight loss due to increased patient mobility, etc.).

Similar to above, the timing of the second surgical procedure can be fixed, adaptive, or both. For example, the timing can be based on a fixed duration from the date the first surgical procedure is performed. In such embodiments, the fixed duration between the first surgical procedure and the second surgical procedure may be between about 6 months and 10 years, such as about 6 months, 9 months, 12 months, 2 years, 3 years, 5 years, or 10 years. The timing can also be adaptive, in which one or more patient metrics are tracked following the first surgical procedure. Once the tracked metrics meet a particular threshold, the patient is ready for the second surgical procedure.

There can also be additional operations associated with determining the second treatment stage. For example, determining the second treatment stage can include identifying or designing a second target anatomical configuration, similar to how determining the first treatment stage can include designing a first target anatomical configuration. The second target anatomical configuration can reflect a desired and/or predicted anatomy of the patient if the second treatment stage were to be performed as prescribed. One or more virtual models can be generated that show the second target anatomical configuration. In some embodiments, the second corrected anatomical configuration is identified/determined before the second surgical procedure and/or the second target location. That is, a computing system or user can model a preferred anatomical outcome, and, based on the desired anatomical outcome, identify the second surgical procedure and the second target location that will achieve the desired anatomical outcome once performed. Patient metrics associated with the second corrected anatomical configuration can also be calculated.

As a non-limiting example, virtual models can include a visual representation of the patient's spinal cord region, including some or all of the sacrum, lumbar region, thoracic region, and/or cervical region, in a corrected anatomical configuration. The virtual models can be designed to incorporate expected anatomical changes associated with previous treatment stages. For example, if the first treatment stage includes a corrective intervention to the lumbar region and the second treatment stage includes a corrective intervention to the cervical region, the virtual model generated for the second treatment stage can include the expected anatomical corrections that will occur during the first treatment stage. The second treatment stage can therefore be planned using a virtual model that incorporates any anatomical corrections that may occur during the first stage. Without being bound by theory, this is expected to improve planning of the second treatment stage because an anatomical change during the first treatment stage may affect the interventions planned during the second treatment stage (e.g., lumbar correction can affect cervical alignment). As set for above, the multi-stage surgical plan can also include planned metrics for each stage based on stage-specific virtual models.

Although the foregoing describes determining the first treatment stage and the second treatment stage sequentially, one skilled in the art will appreciate from the disclosure herein that the medical treatment determined for the second treatment stage is based at least in part on the medical treatment determined for the first treatment stage, and vice versa. For example, the first surgical procedure may be reduced in invasiveness, complexity, expected recovery time, or the like because of the treatment prescribed to be given during the second treatment stage. Accordingly, the first surgical procedure may be different than a surgical procedure that would have been prescribed without the expectation of the second surgical procedure. Similarly, the second surgical procedure may be based on a patient's most likely long-term outcome following the first surgical procedure, and/or to address an altered progression of a patient's condition caused by the first surgical procedure. For example, if the first surgical procedure specifies a first particular surgical approach (e.g., lateral, posterior, anterior, etc.), the second surgical procedure may purposefully have a different surgical approach, e.g., to minimize potential interference by scar tissue and/or to reduce repeated trauma to the same tissue.

Moreover, in some embodiments, the computing systems used to generate the first treatment stage and the second treatment stage can also predict whether a particular surgical procedure may induce a change in patient anatomy at a location other than the first target location. For example, the system may determine by analyzing reference patient data and/or by analyzing the virtual model of patient anatomy that a first surgical procedure that fuses two or more sacral vertebral levels may induce misalignment at one or more cervical vertebral levels, reduce mobility at one or more thoracic vertebral levels, or the like. Systems and methods for predicting anatomical effects of surgical interventions at locations spaced apart from the target surgical location are described in U.S. Patent Application No. 63/437,975, the disclosure of which is incorporated by reference herein in its entirety. The second treatment stage can be designed based specifically on these predicted changes at locations other than the first target location. That is, the second treatment stage not only can account for anticipated changes at the first target location, but also can account for predicted changes at anatomical locations spaced apart from the first target location. Indeed, the second treatment stage may be designed to address one or more changes at anatomical locations spaced apart from the first target location (e.g., the second surgical procedure may correct cervical misalignment, improve thoracic mobility, etc.).

The order of the first treatment stage and the second treatment stage can also be selected specifically based on computer modeling of patient response to the first surgical procedure and the second surgical procedure. For example, certain surgical procedures described herein involve implant insertion, disc resection, bony resection, pedicle screw insertions, and other invasive measures that change patient anatomy. Depending on the particular surgical interventions that are prescribed, it may be advantageous to perform certain procedures before others. For example, if pedicle screw insertion during a first particular procedure may impact the ability to perform a second particular surgical procedure, the second particular surgical procedure may be prescribed to be performed before the first surgical procedure. Such “interferences” between surgical procedures can be identified by modeling patient responses to various treatment stages using virtual models of patient anatomy, as described throughout this Detailed Description.

Accordingly, the method 400 is expected provide a comprehensive and holistic treatment plan for a particular patient over an extended time period (e.g., at least 5 years, at least 10 years, at least 20 years, etc.). Without being bound by theory, this is expected to (1) provide better patient outcomes by optimizing not only the type of medical treatment the patient receives, but also the timing and the order the patient receives it, and (2) provide greater transparency to the patient, surgeon, and other healthcare providers about current and future medical treatments that the patient will need.

Once the first treatment stage and the second treatment stage are generated at blocks 404 and 406, the method 400 can continue at block 408 by preparing a multi-stage surgical plan. The multi-stage surgical plan can include the first treatment stage and the second treatment stage. In some embodiments, the multi-stage surgical plan can include additional treatment stages, for a total of three, four, five, six, or more treatment stages. Each treatment stage can include a surgical procedure or other medical intervention and a recommended time for performing the surgical procedure or other medical intervention.

The multi-stage surgical plan prepared at block 408 can also include additional features. In some embodiments, for example, the multi-stage surgical plan can include predicted outcomes for the first treatment stage and the second treatment stage and probabilities of success for achieving the predicted outcomes. In such embodiments, the method can include defining a successful outcome for each of the treatment stages (e.g., by selecting patient metrics associated with a successful outcome) and calculating a probability that each of the stages will have a successful outcome. The multi-stage plan can also include predicted disease progression, predicted patient satisfaction, predicted patient mobility, predicted patient pain, predicted patient quality of life, or the like at and/or after various stages of the multi-stage treatment plan. For example, the surgical plan may include estimates of disease progression if the patient were to undergo the first treatment stage and the second treatment stage. In such embodiments, the surgical plan can include virtual models (e.g., two-dimensional or three-dimensional virtual models) of patient anatomy at various intervals after the first treatment stage and/or the second treatment stage. For example, the surgical plan may include a predictive model of patient anatomy at 6 months, 1 year, 2 years, 3 years, 4 years, 5 years, and/or 10 years after the first treatment stage and/or the second treatment stage. The disease progression model may also include predicted patient metrics (e.g., any of the patient metrics described herein, including coronal parameters, sagittal parameters, pelvic parameters, Cobb angles, shoulder tilt, iliolumbar angles, coronal balance, lordosis angles, intervertebral space height, or other similar spinal parameters) at any of the various post-operative intervals identified above, in addition to or in lieu of including the virtual model of predicted patient anatomy. Similarly, the surgical plan can include probabilities associated with projected progression, e.g., to account for unexpected patient responses to the first surgical procedure, and/or to account for potential unpredicted lifestyle changes by the patient (e.g., weight gain).

In some embodiments, the multi-stage surgical plan can be generated with multiple alternative treatment stages based on previous treatment stages. For example, the multi-stage surgical plan can be generated with multiple alternative second treatment stages. In such embodiments, the multi-stage plan can include metrics for selecting the alternative treatment stages. For example, the multi-stage plan can include a second treatment stage “option A” that should be selected if a patient metric meets a particular threshold (e.g., disc height greater than 10 mm at a particular vertebral level, prevalence of scar tissue, spinal mobility, patient weight, patient pain level, patient disability score, etc.), and a second treatment stage “option B” that should be selected if the patient metric does not meet the particular threshold by the time the second treatment stage is set to begin. Therefore, the multi-stage plan can include selection criteria for selecting future treatment stages based on outcomes from a prior stage, which can be selected before the first treatment stage occurs. In some embodiments, a computing system can analyze reference patient data to automatically identify and set one or more of the selection criteria. This allows for adaptive customization of treatment based on outcomes of each stage.

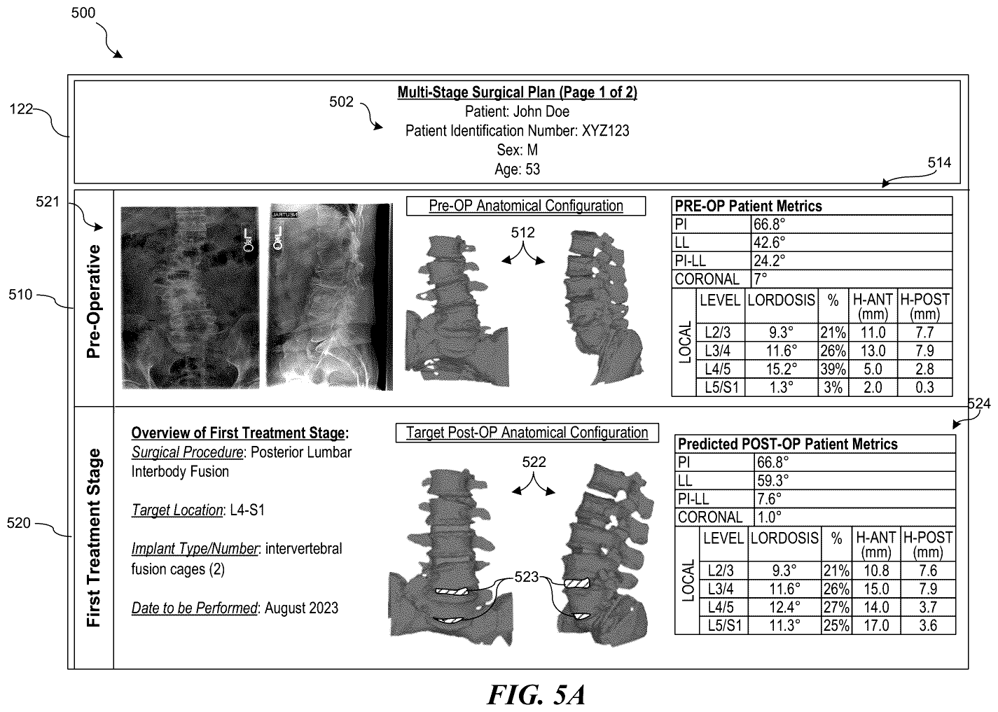

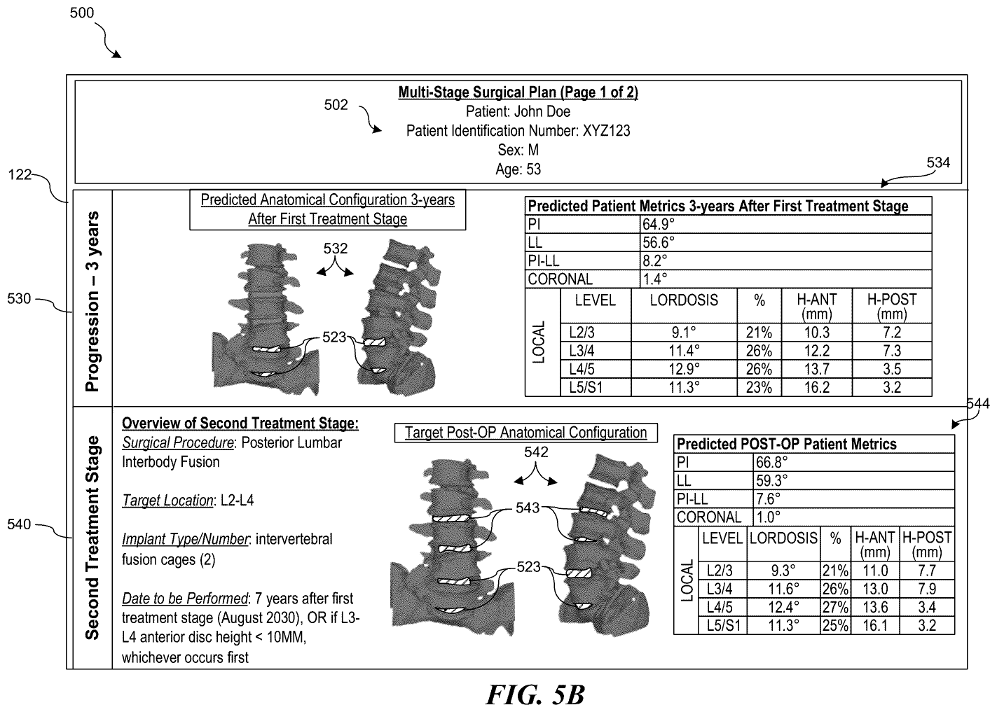

In some embodiments, preparing the multi-stage surgical plan can include generating a multi-stage surgical plan report. This can include, for example, generating instructions encoding the multi-stage surgical plan that, when executed by a corresponding computing program (e.g., the review module 123 of FIG. 1), cause aspects of the multi-stage surgical plan to be displayed for user review (e.g., via the surgical plan review program 125 of FIG. 1). An example of a representative multi-stage surgical plan report is described below with reference to FIGS. 5A and 5B.

Once the multi-stage surgical plan is prepared at block 408, the method 400 can continue at block 410 by transmitting the multi-stage surgical plan to a surgeon for review. The operation performed at block 410 can be the same as, or at least generally similar to, the operation performed at block 306 of the method 300 described with reference to FIG. 3. The method 400 can optionally be followed by the operations described at blocks 308-318 of the method 300.

In some embodiments, the computing system used to generate the multi-stage plan or another suitable computing system can receive updates as the multi-stage plan is being performed. For example, a computing system may receive patient metrics associated with a patient's actual outcome to the first treatment stage. The system can analyze whether the multi-stage plan should proceed as designed, or if any modifications to the plan are needed. As a first example, a patient may experience more subsidence than predicted following the first treatment stage, thereby indicating poor bone tissue (e.g., weakened endplates of vertebral bodies). Planned subsequent stages can be modified to compensate for such patient-specific pathology. As a second example, a patient may experience more scar tissue than predicted following the first treatment stage, thereby indicating poor response to a particular surgical approach. Planned subsequent stages can be modified to utilize a different surgical approach. On the other hand, if the patient experiences less scar tissue than predicted following the first treatment stage, thereby indicating a favorable response to a particular surgical approach, planned subsequent stages can be modified to utilize the same surgical approach. The system can also analyze whether any unexpected anatomical effects occurred at locations apart from the first target location (e.g., cervical misalignment following lumbar fusion), and if so, if any adjustments to the second treatment stage are needed to compensate for the unexpected anatomical effects.

Accordingly, if planned pathology corrections are not achieved by a stage, subsequent stages can be modified or added to help achieve the planned pathology corrections. In such embodiments, the computing system can update and/or generate new virtual models of the patient's current anatomy and incorporating updated subsequent stages that are based on actual outcomes from one or more previous stages. For example, tissue characteristics (e.g., bone strength, fracture toughness, scar tissue, etc.) of virtual models can be based on outcomes from completed stages, as previously described. As previously described, in embodiments in which the multi-stage plan includes a plurality of alternative second treatment stages, the system can also analyze the actual outcomes of the first treatment stage to determine which of a plurality of alternative second treatment stages should be performed. Accordingly, the methods and systems described herein can repeatedly analyze the interrelationships between treatment stages, pathological conditions, treatment stage outcomes, patient feedback (e.g., pain scores), and other data.

### C. Representative Multi-Stage Surgical Plans

FIGS. 5A and 5B illustrate a representative multi-stage surgical plan 500 (“the plan 500”) displayed via the display 122 in accordance with embodiments of the present technology. In particular, FIG. 5A illustrates a first portion of the plan 500, and FIG. 5B illustrates a second portion of the plan 500. Although shown separately, one skilled in the art will appreciate that the first portion and second portion can optionally be displayed side-by-side or as part of a continuous (e.g., scrollable) page, depending on the size and features of the display 122.

Referring collective to FIGS. 5A and 5B, the plan 500 includes patient data 502. In the illustrated embodiment, the patient data includes patient name, patient identification number (e.g., MRN), sex, and age, although other patient data can be included in addition to or in lieu of the foregoing data. The plan 500 also includes discrete sections associated with various stages of the plan 500. For example, the plan 500 includes a pre-operative section 510 with associated pre-operative data, a first treatment stage section 520 with associated first treatment stage data, a 3-year progression section 530 with associated disease progression data, and a second treatment stage section 540 with associated second treatment stage data.

The pre-operative section 510, shown in FIG. 5A, can include patient images 521 showing pre-operative patient anatomy. Although shown as radiographic images, in other embodiments other image types may be included (e.g., MRI), or the pre-operative images 521 may be omitted. The plan 500 further includes a virtual model 512 showing a pre-operative (e.g., native) anatomical configuration of the patient (e.g., a “pre-operative virtual model 512”), along with pre-operative patient metrics 514. In the illustrated embodiment, the pre-operative virtual model 512 is a three-dimensional model showing the patient's lumbar spinal region, although in other embodiments the pre-operative virtual model 512 can be two-dimensional and include more or less patient anatomy.

The first treatment stage section 520, also shown in FIG. 5A, includes an overview of the first treatment stage. For example, the section 520 may include a surgical procedure to be performed as part of the first treatment stage, a target location for the surgical procedure, a description of any implants or other devices to be implanted or otherwise used during the surgical procedure, and a recommended date to perform the surgical procedure. The foregoing data can be determined as described above with respect to the method 400 of FIG. 4. The first treatment stage section 520 can also include a post-operative virtual model 522 showing predicted post-operative anatomical configuration of the patient. The post-operative virtual model 522 can optionally show one or more virtual implants 523 implanted at the target locations. The first treatment stage section 520 can also include post-operative patient metrics 524, which are predicted patient metrics for the patient if the first treatment stage is performed.

The 3-year progression section 530, shown in FIG. 5B, can include data associated with how the patient's condition is expected to change over time if the first surgical procedure were to be performed during the first treatment stage. In particular, the section 530 can include a virtual model 532 showing predicted patient anatomy 3 years after the first surgical procedure and patient metrics 534 associated with the virtual model 532. Accordingly, the section 530 provides an estimate of how a patient's condition may change over time following the first treatment stage. Although shown as modeling patient anatomy and associated metrics at 3-years after the first treatment stage, the plan 500 can include estimates at any time period following the first treatment stage, such as 6-months, 1-year, 2-years, 3-years, 4-years, 5-years, 10-years, etc. Moreover, the plan 500 can include more than one progression model. For example, the plan 500 can include predicted patient anatomy and associated metrics at defined, discrete intervals beginning after the first treatment stage (e.g., 6-month increments, yearly increments, etc.). The plan 500 can also include progression estimates that may predate the first treatment stage or follow the second treatment stage. The data shown in section 530 can be generated using, for example, the disease progression module 120 described with reference to FIG. 1.

The second treatment stage section 540, also shown in FIG. 5B, includes data generally similar to the first treatment stage section 520 but for the second treatment stage. For example, the section 540 may include a surgical procedure to be performed as part of the second treatment stage, a target location for the surgical procedure, a description of any implants or other devices to be implanted or otherwise used during the surgical procedure, and recommended timing to perform the surgical procedure. The foregoing data can be determined as described above with respect to the method 400 of FIG. 4. The second treatment stage section 540 can also include a post-operative virtual model 542 showing a predicted post-operative anatomical configuration of the patient for if the second treatment stage is performed. The post-operative virtual model 542 can optionally show one or more virtual implants 543 implanted at the target locations. Lastly, the second treatment stage section 540 can include post-operative patient metrics 544, which are predicted patient metrics for the patient if the second treatment stage is performed.

The plan 500 is provided by way of example only. One skilled in the art will appreciate from the disclosure herein that many iterations and variations of multi-stage surgical plans can be generated using the systems and methods of the present technology. Accordingly, the present technology is not limited to the specific representation of the surgical plan provided in FIGS. 5A and 5B, unless clearly expressed otherwise. Moreover, although depicted on the display 122, the plan 500 can be displayed via other suitable display screens, and/or stored via computer-executable instructions on a computing device, server, or other cloud-based storage systems. The plan 500 can also include additional information not shown in FIGS. 5A and 5B, such as additional treatment stages, additional disease progression modeling, physician information (e.g., physician-specific expertise, physician-specific prior surgical outcomes, etc.), regulatory information (e.g., regulatory requirements, regulatory-based reimbursements information, etc.), insurance or payment information (e.g., coverage for some or all of the surgical plan), reimbursement criteria, healthcare/provider expertise, available surgical equipment, manufacturing capabilities, combinations thereof, or the like.

### D. Additional Select Methods and Systems for Modeling and Designing Multi-Stage Surgical Plans

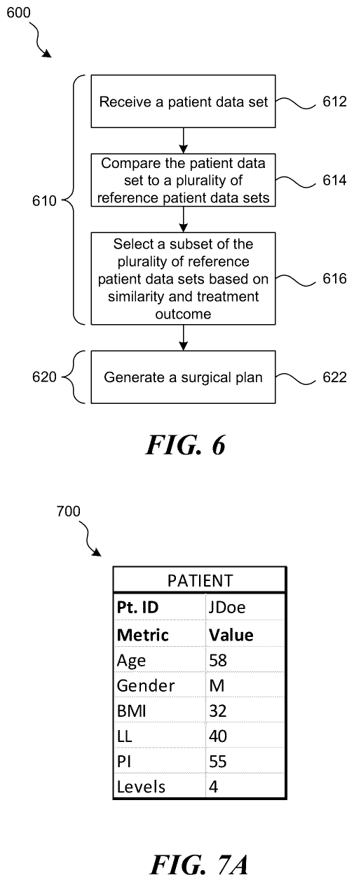

FIG. 6 is a flow diagram illustrating a method 600 for generating a surgical plan, such as a multi-stage surgical plan, according to an embodiment of the present technology. In some embodiments, the method 600 is performed at blocks 302 and 304 of the method 300 described previously. The method 600 can include a data phase 610 and a modeling phase 620. The data phase 610 can include collecting data of a patient to be treated (e.g., pathology data), and comparing the patient data to reference data (e.g., prior patient data such as pathology, surgical, and/or outcome data). For example, a patient data set can be received (block 612). The patient data set can be compared to a plurality of reference patient data sets (block 614), e.g., in order to identify one or more similar patient data sets in the plurality of reference patient data sets. Each of the plurality of reference patient data sets can include data representing one or more of age, gender, BMI, lumbar lordosis, Cobb angle(s), pelvic incidence, disc height, segment flexibility, bone quality, rotational displacement, or treatment level of the spine.

A subset of the plurality of reference patient data sets can be selected (block 616), e.g., based on similarity to the patient data set and/or treatment outcomes of the corresponding reference patients. For example, a similarity score can be generated for each reference patient data set, based on the comparison of the patient data set and the reference patient data set. The similarity score can represent a statistical correlation between the patient data and the reference patient data set. One or more similar patient data sets can be identified based, at least partly, on the similarity score.

In some embodiments, each patient data set of the selected subset includes and/or is associated with data indicative of a favorable treatment outcome (e.g., a favorable treatment outcome based on a single target outcome, aggregate outcome score, outcome thresholding). The data can include, for example, data representing one or more of corrected anatomical metrics, presence of fusion, health related quality of life, activity level, or complications. In some embodiments, the data is or includes an outcome score, which can be calculated based on a single target outcome, an aggregate outcome, and/or an outcome threshold.

Optionally, the data analysis phase 610 can include identifying or determining, for at least one patient data set of the selected subset (e.g., for at least one similar patient data set), surgical procedure data and/or medical device design data associated with the favorable treatment outcome. The surgical procedure data can include data representing one or more of a surgical approach, a corrective maneuver, a bony resection, or implant placement. The at least one medical device design can include data representing one or more of physical properties, mechanical properties, or biological properties of a corresponding medical device. In some embodiments, the at least one patient-specific medical device design includes a design for an implant or an implant delivery instrument.

In the modeling phase 620, a surgical plan (e.g., a multi-stage surgical plan) is generated (block 622). The generating step can include developing at least one predictive model based on the patient data set and/or selected subset of reference patient data sets (e.g., using statistics, machine learning, neural networks, AI, or the like). The predictive model can be configured to generate the surgical plan. The surgical plan can be generally similar to any of the surgical plans described herein, including the multi-stage surgical plans described above.

In some embodiments, the predictive model includes one or more trained machine learning models that generate, at least partly, the surgical plan. For example, the trained machine learning model(s) can determine a plurality of candidate surgical plans for treating the patient. Each surgical plan can be associated with a corresponding medical device design. In some embodiments, the surgical plans and/or medical device designs are determined based on surgical procedure data and/or medical device design data associated with favorable outcomes, as previously described with respect to the data analysis phase 610. For each surgical plan and/or corresponding medical device design, the trained machine learning model(s) can calculate a probability of achieving a target outcome (e.g., favorable or desired outcome) for the patient. The trained machine learning model(s) can then select one or more surgical plans and/or corresponding medical device designs based, at least partly, on the calculated probabilities.

The method 600 can be implemented and performed in various ways. In some embodiments, one or more operations of the method 600 (e.g., the data phase 610 and/or the modeling phase 620) can be implemented as computer-readable instructions stored in memory and executable by one or more processors of any of the computing devices and systems described herein (e.g., the system 100), or a component thereof (e.g., the client computing device 102 and/or the server 106). Alternatively, one or more operations of the method 600 can be performed by a healthcare provider (e.g., physician, surgeon), a robotic apparatus (e.g., a surgical robot), or a combination thereof.

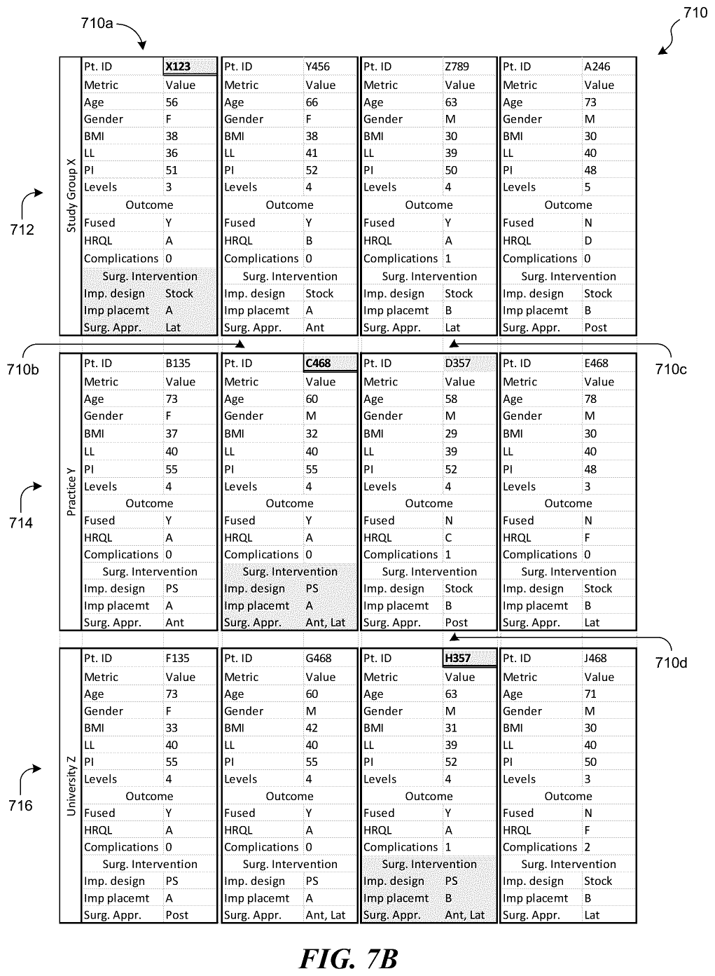

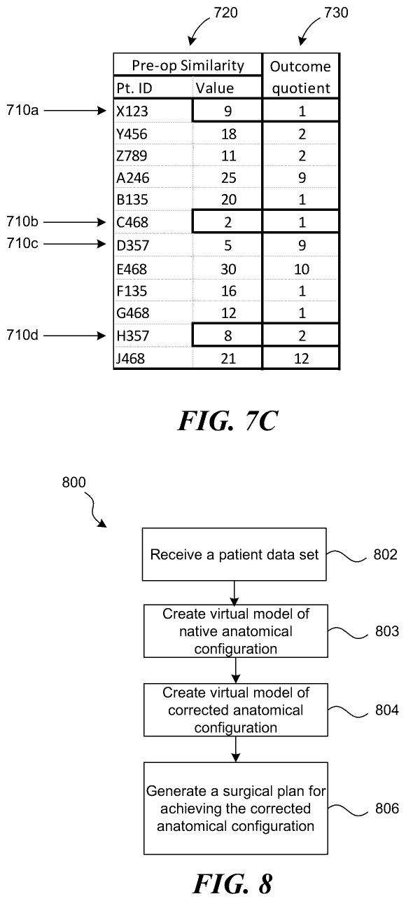

FIGS. 7A-7C illustrate exemplary data sets that may be used and/or generated in connection with the methods described herein (e.g., the data analysis phase 610 described with respect to FIG. 6), according to an embodiment of the present technology. FIG. 7A illustrates a patient data set 700 of a patient to be treated. The patient data set 700 can include a patient ID and a plurality of pre-operative patient metrics (e.g., age, gender, BMI, lumbar lordosis (LL), pelvic incidence (PI), and treatment levels of the spine (levels)). FIG. 7B illustrates a plurality of reference patient data sets 710. In the depicted embodiment, the reference patient data sets 710 include a first subset 712 from a study group (Study Group X), a second subset 717 from a practice database (Practice Y), and a third subset 716 from an academic group (University Z). In alternative embodiments, the reference patient data sets 710 can include data from other sources, as previously described herein. Each reference patient data set can include a patient ID, a plurality of pre-operative patient metrics (e.g., age, gender, BMI, lumbar lordosis (LL), pelvic incidence (PI), and treatment levels of the spine (levels)), treatment outcome data (Outcome) (e.g., presence of fusion (fused), HRQL, complications), and treatment procedure data (Surg. Intervention) (e.g., implant design, implant placement, surgical approach).

FIG. 7C illustrates comparison of the patient data set 700 to the reference patient data sets 710. As previously described, the patient data set 700 can be compared to the reference patient data sets 710 to identify one or more similar patient data sets from the reference patient data sets. In some embodiments, the patient metrics from the reference patient data sets 710 are converted to numeric values and compared the patient metrics from the patient data set 700 to calculate a similarity score 720 (“Pre-op Similarity”) for each reference patient data set. Reference patient data sets having a similarity score below a threshold value can be considered to be similar to the patient data set 700. For example, in the depicted embodiment, reference patient data set 710a has a similarity score of 9, reference patient data set 710b has a similarity score of 2, reference patient data set 710c has a similarity score of 5, and reference patient data set 710d has a similarity score of 8. Because each of these scores are below the threshold value of 10, reference patient data sets 710a-d are identified as being similar patient data sets.

The treatment outcome data of the similar patient data sets 710a-d can be analyzed to determine surgical plans and/or implant designs with the highest probabilities of success. For example, the treatment outcome data for each reference patient data set can be converted to a numerical outcome score 730 (“Outcome Quotient”) representing the likelihood of a favorable outcome. In the depicted embodiment, reference patient data set 710a has an outcome score of 1, reference patient data set 710b has an outcome score of 1, reference patient data set 710c has an outcome score of 9, and reference patient data set 710d has an outcome score of 2. In embodiments where a lower outcome score correlates to a higher likelihood of a favorable outcome, reference patient data sets 710a,710b, and 710d can be selected. The treatment procedure data from the selected reference patient data sets 710a, 710b, and 710d can then be used to determine at least one surgical plan (e.g., implant placement, surgical approach) and/or implant design that is likely to produce a favorable outcome for the patient to be treated.

In some embodiments, a method for providing medical care to a patient is provided. The method can include comparing a patient data set to reference data. The patient data set and reference data can include any of the data types described herein. The method can include identifying and/or selecting relevant reference data (e.g., data relevant to treatment of the patient, such as data of similar patients and/or data of similar treatment procedures), using any of the techniques described herein. A surgical plan can be generated based on the selected data, using any of the techniques described herein. The surgical plan can include one or more treatment procedures (e.g., surgical procedures, instructions for procedures, models or other virtual representations of procedures), one or more medical devices (e.g., implanted devices, instruments for delivering devices, surgical kits), or a combination thereof.

In some embodiments, a system for generating a medical treatment plan is provided. The system can compare a patient data set to a plurality of reference patient data sets, using any of the techniques described herein. A subset of the plurality of reference patient data sets can be selected, e.g., based on similarity and/or treatment outcome, or any other technique as described herein. A surgical plan can be generated based at least in part on the selected subset, using any of the techniques described herein. The surgical plan can include one or more treatment procedures, one or more medical devices, or any of the other aspects of a treatment plan described herein, or combinations thereof.

In further embodiments, a system is configured to use historical patient data. The system can select historical patient data to develop or select a surgical plan, design medical devices, or the like. Historical data can be selected based on one or more similarities between the present patient and prior patients to develop a prescriptive treatment plan designed for desired outcomes. The prescriptive treatment plan can be tailored for the present patient to increase the likelihood of the desired outcome. In some embodiments, the system can analyze and/or select a subset of historical data to generate one or more surgical procedures, one or more medical devices, or a combination thereof. In some embodiments, the system can use subsets of data from one or more groups of prior patients, with favorable outcomes, to produce a reference historical data set used to, for example, design, develop or select the treatment plan, medical devices, or combinations thereof.

FIG. 8 is a flow diagram illustrating another method 800 for providing patient-specific medical care, according to another embodiment of the present technology. Similar to the method 600 described with reference to FIG. 6, the method 800 can be performed at blocks 302 and 304 of the method 300 described previously.

The method 800 can begin in block 802 by receiving a patient data set for a particular patient in need of medical treatment. In some embodiments, block 802 can be the same as or generally similar to the block 302 of the method 300 of FIG. 3A. For example, receiving a patient data set can include receiving patient image data, patient metrics, or any of the other patient data described herein.

Once the patient data set is received in block 802, the method 800 can continue in block 803 by creating a virtual model of the patient's native anatomical configuration (also referred to as “pre-operative anatomical configuration”). The virtual model can be based on the image data included in the patient data set received in block 802. For example, the same computing system that received the patient data set in block 802 can analyze the image data in the patient data set to generate a virtual model of the patient's native anatomical configuration. The virtual model can be a two- or three-dimensional visual representation of the patient's native anatomy. The virtual model can include one or more regions of interest, and may include some or all of the patient's anatomy within the regions of interest (e.g., any combination of tissue types including, but not limited to, bony structures, cartilage, soft tissue, vascular tissue, nervous tissue, etc.). As a non-limiting example, the virtual model can include a visual representation of the patient's spinal cord region, including some or all of the sacrum, lumbar region, thoracic region, and/or cervical region. In some embodiments, the virtual model includes soft tissue, cartilage, and other non-bony structures. In other embodiments, the virtual model only includes the patient's bony structures. In some embodiments, the method 800 can optionally omit creating a virtual model of the patient's native anatomy in block 803, and proceed directly from block 802 to block 804.

In some embodiments, the computing system that generated the virtual model in block 802 can also determine (e.g., automatically determine or measure) one or more disease metrics of the patient based on the virtual model. For example, the computing system may analyze the virtual model to determine the patient's pre-operative lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine).

The method 800 can continue in block 804 by creating a virtual model of a corrected anatomical configuration (which can also be referred to herein as the “planned configuration,” “optimized geometry,” “post-operative anatomical configuration,” or “target outcome”) for the patient. For example, the computing system can, using the analysis procedures described previously, determine a “corrected” or “optimized” anatomical configuration for the particular patient that represents an ideal surgical outcome for the particular patient. This can be done, for example, by analyzing a plurality of reference patient data sets to identify post-operative anatomical configurations for similar patients who had a favorable post-operative outcome, as previously described in detail with respect to FIGS. 6-7C (e.g., based on similarity of the reference patient data set to the patient data set and/or whether the reference patient had a favorable treatment outcome). This may also include applying one or more mathematical rules defining optimal anatomical outcomes (e.g., positional relationships between anatomic elements) and/or target (e.g., acceptable) post-operative metrics/design criteria (e.g., adjust anatomy so that the post-operative sagittal vertical axis is less than 7 mm, the post-operative Cobb angle less than 10 degrees, etc.). Target post-operative metrics can include, but are not limited to, target coronal parameters, target sagittal parameters, target pelvic incidence angle, target Cobb angle, target shoulder tilt, target iliolumbar angle, target coronal balance, target Cobb angle, target lordosis angle, and/or a target intervertebral space height. The difference between the native anatomical configuration and the corrected anatomical configuration may be referred to as a “patient-specific correction” or “target correction.”

Once the corrected anatomical configuration is determined, the computing system can generate a two- or three-dimensional visual representation of the patient's anatomy with the corrected anatomical configuration. As with the virtual model created in block 803, the virtual model of the patient's corrected anatomical configuration can include one or more regions of interest, and may include some or all of the patient's anatomy within the regions of interest (e.g., any combination of tissue types including, but not limited to, bony structures, cartilage, soft tissue, vascular tissue, nervous tissue, etc.). As a non-limiting example, the virtual model can include a visual representation of the patient's spinal cord region in a corrected anatomical configuration, including some or all of the sacrum, lumbar region, thoracic region, and/or cervical region. In some embodiments, the virtual model includes soft tissue, cartilage, and other non-bony structures. In other embodiments, the virtual model only includes the patient's bony structures.

The method 800 can continue in block 806 by generating (e.g., automatically generating) a surgical plan for achieving the corrected anatomical configuration shown by the virtual model. The surgical plan can be generally similar to the surgical plans described herein, and can include, for example, multiple treatment stages, pre-operative plans, operative plans, post-operative plans, and/or specific spine metrics associated with the optimal surgical outcome. For example, the surgical plans can include a specific surgical procedure for achieving the corrected anatomical configuration. In the context of spinal surgery, the surgical plan may include a specific fusion surgery (e.g., PLIF, ALIF, TLIF, LLIF, DLIF, XLIF, etc.) across a specific range of vertebral levels (e.g., L1-L4, L1-5, L3-T12, etc.). Other surgical procedures may also be identified for achieving the corrected anatomical configuration, such as non-fusion surgical approaches and orthopedic procedures for other areas of the patient. The surgical plan may also include one or more expected spine metrics (e.g., lumbar lordosis, Cobb angles, coronal parameters, sagittal parameters, and/or pelvic parameters) corresponding to the expected post-operative patient anatomy, as previously described. The surgical plan can be generated by the same or different computing system that created the virtual model of the corrected anatomical configuration. In some embodiments, the surgical plan can also be based on one or more reference patient data sets as previously described with respect to FIGS. 6-7C. In some embodiments, the surgical plan can also be based at least in part on surgeon-specific preferences and/or outcomes associated with a specific surgeon performing the surgery.

FIG. 9 is a schematic illustration of an operative setup including select systems and devices that can be used to provide patient-specific medical care, such as for performing certain operations described above with respect to the methods 300, 400, 600, and 800, described with respect to FIGS. 3, 4, 6, and 8, respectively. As shown, the operative setup includes a computing device 902, a computing system 906, a cloud 908, a manufacturing system 930, and a robotic surgical platform 950. The computing device 902 can be a user device, such as a smart phone, mobile device, laptop, desktop, personal computer, tablet, phablet, or other such devices known in the art. In operation, a user (e.g., a surgeon) can collect, retrieve, review, modify, or otherwise interact with a patient data set using the computing device 902. The computing system 906 can include any suitable computing system configured to store one or more software modules for identifying reference patient data sets, determining patient-specific surgical plans, generating virtual models of patient anatomy, designing patient-specific implants, or the like. The one or more software modules can include algorithms, machine-learning models, artificial intelligence architectures, or the like for performing select operations. The cloud 908 can be any suitable network and/or storage system, and may include any combination of hardware and/or virtual computing resources. The manufacturing system 930 can be any suitable manufacturing system for producing patient-specific implants, including any of those previously described herein. The robotic surgical platform 950 (referred to herein as “the platform 950”) can be configured to perform or otherwise assist with one or more aspects of a surgical procedure.

In a representative operation, the computing device 902, the computing system 906, the cloud 908, the manufacturing system 930, and the platform 950 can be used to provide patient-specific medical care, such as to perform the methods described herein. For example, the computing system 906 can receive a patient data set from the computing device 902 (e.g., block 302 of the method 300, block 802 of the method 800, etc.). In some embodiments, the computing device 902 can directly transmit the patient data set to the computing system 906. In other embodiments, the computing device 902 can upload the patient data set into the cloud 908, and the computing system 906 can download or otherwise access the patient data set from the cloud. Once the computing system 906 receives the patient data set, the computing system 906 can create a virtual model of the patient's native anatomical configuration (e.g., block 803 of the method 800), create a virtual model of the corrected anatomical configuration (e.g., block 804 of the method 800), and/or generate a surgical plan for achieving the corrected anatomical configuration (e.g., block 304 of the method 300, block 408 of the method 400, block 806 of the method 800, etc.). The computing system can perform the foregoing operations via the one or more software modules, which in some embodiments include machine learning models or other artificial intelligence architectures. Once the surgical plans, including any associated virtual models, are created, the computing system 906 can transmit the surgical plans to the surgeon for review (e.g., block 306 of the method 300, block 410 of the method 400). This can include, for example, directly transmitting the surgical plans to the computing device 902 for surgeon review. In other embodiments, this can include uploading the virtual models and the surgical plan to the cloud 908. A surgeon can then download or otherwise access the virtual models and the surgical plan from the cloud 908 using the computing device 902.

The surgeon can use the computing device 902 to review the surgical plans, as described previously. The surgeon can also approve or reject the surgical plans and provide any feedback regarding the surgical plans using the computing device 902. The surgeon's approval, rejection, and/or feedback regarding the surgical plan can be transmitted to, and received by, the computing system 906 (e.g., blocks 308 and 310 of the method 300). The computing system 906 can than revise the virtual surgical plans and associated virtual models (e.g., block 312 of the method 300). The computing system 906 can transmit the revised virtual model and surgical plan to the surgeon for review (e.g., by uploading it to the cloud 908 or directly transmitting it to the computing device 902).

The computing system 906 can also design the patient-specific implant based on the selected surgical plan (e.g., block 314 of the method 300) using, the one or more software modules. In some embodiments, software modules rely on one or more algorithms, machine learning models, or other artificial intelligence architectures to design the implant. Once the computing system 906 designs the patient-specific implant, the computing system 906 can upload the design and/or manufacturing instructions to the cloud 908. The computing system 906 may also create fabrication instructions (e.g., computer-readable fabrication instructions) for manufacturing the patient-specific implant. In such embodiments, the computing system 906 can upload the fabrication instructions to the cloud 908.

The manufacturing system 930 can download or otherwise access the design and/or fabrication instructions for the patient-specific implant from the cloud 908. The manufacturing system can then manufacture the patient-specific implant (e.g., block 316 in the method 300) using additive manufacturing techniques, subtractive manufacturing techniques, or other suitable manufacturing techniques.

The robotic surgical platform 950 can perform or otherwise assist with one or more aspects of the surgical procedure (e.g., block 318 of the method 300). For example, the platform 950 can prepare tissue for an incision, make an incision, make a resection, remove tissue, manipulate tissue, perform a corrective maneuver, deliver the implant to a target site, deploy the implant at the target site, adjust the implant at the target site, manipulate the implant once it is implanted, secure the implant at the target site, explant the implant, suture tissue, etc. The platform 950 can therefore include one or more arms 955 and end effectors for holding various surgical tools (e.g., graspers, clips, needles, needle drivers, irrigation tools, suction tools, staplers, screw driver assemblies, etc.), imaging instruments (e.g., cameras, sensors, etc.), and/or medical devices (e.g., the implant 900) and that enable the platform 950 to perform the one or more aspects of the surgical plan. Although shown as having one arm 955, one skilled in the art will appreciate that the platform 950 can have a plurality of arms (e.g., two, three, four, or more) and any number of joints, linkages, motors, and degrees of freedom. In some embodiments, the platform 950 may have a first arm dedicated to holding one or more imaging instruments, while the remainder of the arms hold various surgical tools. In some embodiments, the tools can be releasably secured to the arms such that they can be selectively interchanged before, during, or after an operative procedure. The arms can be moveable through a variety of ranges of motion (e.g., degrees of freedom) to provide adequate dexterity for performing various aspects of the operative procedure.

The platform 950 can include a control module 960 for controlling operation of the arm(s) 955. In some embodiments, the control module 960 includes a user input device (not shown) for controlling operation of the arm(s) 955. The user input device can be a joystick, a mouse, a keyboard, a touchscreen, an infrared sensor, a touchpad, a wearable input device, a camera- or image-based input device, a microphone, or other user input devices. A user (e.g., a surgeon) can interact with the user input device to control movement of the arm(s) 955.

In some embodiments, the control module 960 includes one or more processors for executing machine-readable operative instructions that, when executed, automatically control operation of the arm 955 to perform one or more aspects of the surgical procedure. In some embodiments, the control module 960 may receive the machine-readable operative instructions (e.g., from the cloud 908) specifying one or more steps of the surgical procedure that, when executed by the control module 960, cause the platform 950 to perform the one or more steps of the surgical procedure. For example, the machine-readable operative instructions may direct the platform 950 to prepare tissue for an incision, make an incision, make a resection, remove tissue, manipulate tissue, perform a corrective maneuver, deliver the implant 900 to a target site, deploy the implant 900 at the target site, adjust a configuration of the implant 900 at the target site, manipulate the implant 900 once it is implanted, secure the implant 900 at the target site, explant the implant 900, suture tissue, and the like. The operative instructions may therefor include particular instructions for articulating the arm 955 to perform or otherwise aid in the delivery of the patient-specific implant.

In some embodiments, the platform 950 can generate (e.g., as opposed to simply receiving) the machine-readable operative instructions based on the surgical plan. For example, the surgical plan can include information about the delivery path, tools, and implantation site. The platform 950 can analyze the surgical plan and develop executable operative instructions for performing the patient-specific procedure based on the capabilities (e.g., configuration and number of robotic arms, functionality of and effectors, guidance systems, visualization systems, etc.) of the robotic system. This enables the operative setup shown in FIG. 9 to be compatible with a wide range of different types of robotic surgery systems.

The platform 950 can include one or more communication devices (e.g., components having VLC, WiMAX, LTE, WLAN, IR communication, PSTN, Radio waves, Bluetooth, and/or Wi-Fi operability) for establishing a connection with the cloud 908 and/or the computing device 902 for accessing and/or downloading the surgical plan and/or the machine-readable operative instructions. For example, the cloud 908 can receive a request for a particular surgical plan from the platform 950 and send the plan to the platform 950. Once identified, the cloud 908 can transmit the surgical plan directly to the platform 950 for execution. In some embodiments, the cloud 908 can transmit the surgical plan to one or more intermediate networked devices (e.g., the computing device 902) rather than transmitting the surgical plan directly to the platform 950. A user can review the surgical plan using the computing device 902 before transmitting the surgical plan to the platform 950 for execution. Additional details for identifying, storing, downloading, and accessing patient-specific surgical plans are described in U.S. application Ser. No. 19/960,810, filed Aug. 11, 2020, the disclosure of which is incorporated by reference herein in its entirety.

The platform 950 can include additional components not expressly shown in FIG. 9. For example, in various embodiments the platform 950 may include one or more displays (e.g., LCD display screen, an LED display screen, a projected, holographic, or augmented reality display (e.g., a heads-up display device or a head-mounted device), one or more I/O devices (e.g., a network card, video card, audio card, USB, firewire or other external device, camera, printer, speakers, CD-ROM drive, DVD drive, disk drive, or Blu-Ray device), and/or a memory (e.g., random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth). In some embodiments, the foregoing components can be generally similar to the like components described in detail with respect to computing device 200 in FIG. 2.

Without being bound by theory, using a robotic surgical platform to perform various aspects of the surgical plans described herein is expected to provide several advantages over conventional operative techniques. For example, use of robotic surgical platforms may improve surgical outcomes and/or shorten recovery times by, for example, decreasing incision size, decreasing blood loss, decreasing a length of time of the operative procedure, increasing the accuracy and precision of the surgery (e.g., the placement of the implant at the target location), and the like. The platform 950 can also avoid or reduce user input errors, e.g., by including one or more scanners for obtaining information from instruments (e.g., instruments with retrieval features), tools, the patient specific implant 900 (e.g., after the implant 900 has been gripped by the arm 955), etc. The platform 950 can confirm use of proper instruments prior and during the surgical procedure. If the platform 950 identifies an incorrect instrument or tool, an alert can be sent to a user that another instrument or tool should be installed. The user can scan the new instrument to confirm that the instrument is appropriate for the surgical plan. In some embodiments, the surgical plan includes instructions for use, a list of instruments, instrument specifications, replacement instruments, and the like. The platform 950 can perform pre-and post-surgical checking routines based on information from the scanners.

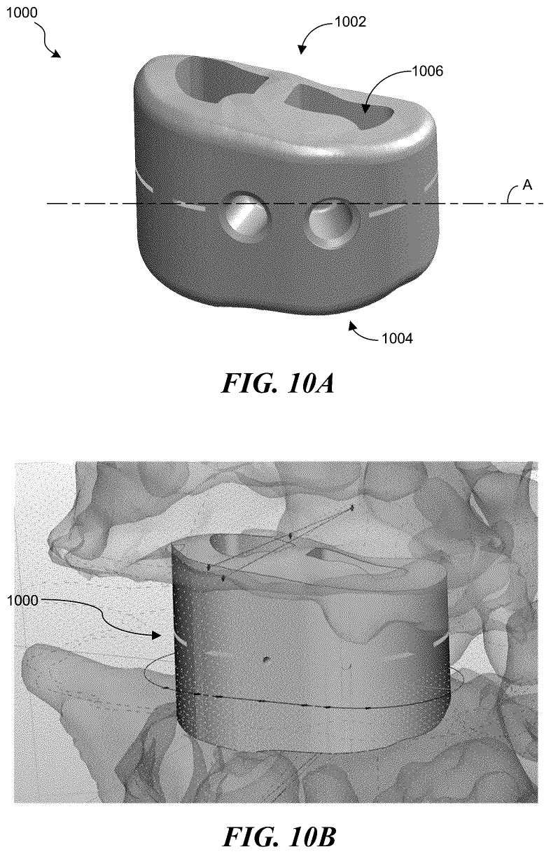

FIG. 10A illustrates an example of a patient-specific implant 1000 (e.g., as designed in block 314 and manufactured in block 316 of the method 300), and FIG. 10B illustrates the implant 1000 implanted in the patient. The implant 1000 can be any orthopedic or other implant specifically designed to induce the patient's body to conform to the previously identified corrected anatomical configuration. In the illustrated embodiment, the implant 1000 is a vertebral interbody device having a first (e.g., upper) surface 1002 configured to engage an inferior endplate surface of a superior vertebral body and a second (e.g., lower) surface 1004 configured to engage a superior endplate surface of an inferior vertebral body. The first surface 1002 can have a patient-specific topography designed to match (e.g., mate with) the topography of the inferior endplate surface of the superior vertebral body to form a generally gapless interface therebetween. Likewise, the second surface 1004 can have a patient-specific topography designed to match or mate with the topography of the superior endplate surface of the inferior vertebral body to form a generally gapless interface therebetween. The implant 1000 may also include a recess 1006 or other feature configured to promote bony ingrowth. Because the implant 1000 is patient-specific and designed to induce a geometric change in the patient, the implant 1000 is not necessarily symmetric, and is often asymmetric. For example, in the illustrated embodiment, the implant 1000 has a non-uniform thickness such that a plane defined by the first surface 1002 is not parallel to a central longitudinal axis A of the implant 1000. Of course, because the implants described herein, including the implant 1000, are patient-specific, the present technology is not limited to any particular implant design or characteristic. Additional features of patient-specific implants that can be designed and manufactured in accordance with the present technology are described in U.S. patent application Ser. Nos. 16/987,111 and 17/100,396, the disclosures of which are incorporated by reference herein in their entireties.

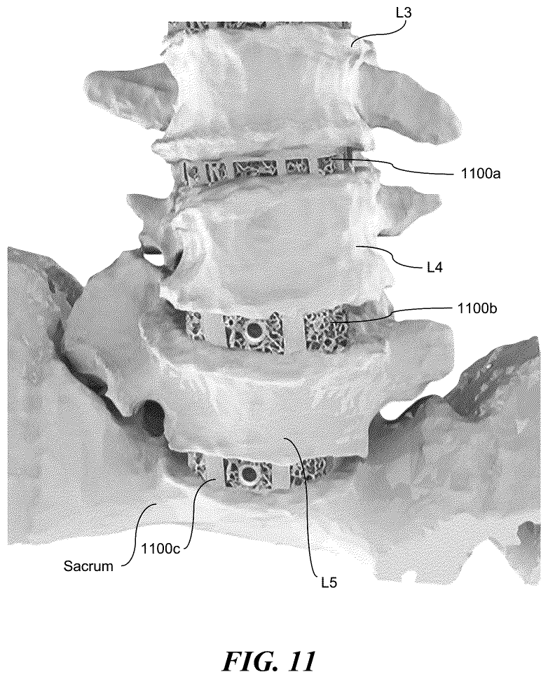

The patient-specific medical procedures described herein can involve implanting more than one patient-specific implant into the patient to achieve the corrected anatomical configuration (e.g., a multi-site procedure). This can include implanting multiple implants during the same procedure (e.g., during a first treatment stage) and/or implanting one or more first implants during a first procedure and one or more second implants during a second procedure. FIG. 11, for example, illustrates a lower spinal cord region having three patient specific implants 1100a-1100c implanted at different vertebral levels. More specifically, a first implant 1100a is implanted between the L3 and L4 vertebral bodies, a second implant 1100b is implanted between the L4 and L5 vertebral bodies, and a third implant 1100c is implanted between the L5 vertebral body and the sacrum. Together, the implants 1100a-c can cause the patient's spinal cord region to assume the previously identified corrected anatomical configuration (e.g., transforming the patient's anatomy from its pre-operative diseased configuration to the post-operative optimized configuration). In some embodiments, more or fewer implants are used to achieve the corrected anatomical configuration. For example, in some embodiments one, two, four, five, six, seven, eight, or more implants are used to achieve the corrected anatomical configuration. In embodiments involving more than one implant, the implants do not necessarily have the same shape, size, or function. In fact, the multiple implants will often have different geometries and topographies to correspond to the target vertebral level at which they will be implanted. As also shown in FIG. 11, the patient-specific medical procedures described herein can involve treating the patient at multiple target regions (e.g., multiple vertebral levels).

### E. Conclusion

As one skilled in the art will appreciate, any of the software modules described previously may be combined into a single software module for performing the operations described herein. Likewise, the software modules can be distributed across any combination of the computing systems and devices described herein, and are not limited to the express arrangements described herein. Accordingly, any of the operations described herein can be performed by any of the computing devices or systems described herein, unless expressly noted otherwise.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2018, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116, filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;” and
U.S. application Ser. No. 16/699,447, filed Nov. 29, 2019, titled “SYSTEMS AND METHODS FOR ORTHOPEDIC IMPLANTS;”
U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS;”
U.S. application Ser. No. 17/100,396, filed Nov. 20, 2020, titled “PATIENT-SPECIFIC VERTEBRAL IMPLANTS WITH POSITIONING FEATURES;”
U.S. application Ser. No. 17/342,439, filed Jun. 8, 2021, titled “PATIENT-SPECIFIC MEDICAL PROCEDURES AND DEVICES, AND ASSOCIATED SYSTEMS AND METHODS;”
U.S. application Ser. No. 17/463,054, filed Aug. 31, 2021, titled “BLOCKCHAIN MANAGED MEDICAL IMPLANTS;”
U.S. application Ser. No. 17/518,524, filed Nov. 3, 2021, titled “PATIENT-SPECIFIC ARTHROPLASTY DEVICES AND ASSOCIATED SYSTEMS AND METHODS;”
U.S. application Ser. No. 17/531,417, filed Nov. 19, 2021, titled “PATIENT-SPECIFIC JIG FOR PERSONALIZED SURGERY;”
U.S. application Ser. No. 17/678,874, filed Feb. 23, 2022, titled “NON-FUNGIBLE TOKEN SYSTEMS AND METHODS FOR STORING AND ACCESSING HEALTHCARE DATA;”
U.S. application Ser. No. 17/835,777, filed Jun. 8, 2022, titled “PATIENT-SPECIFIC EXPANDABLE INTERVERTEBRAL IMPLANTS;”
U.S. application Ser. No. 17/842,242, filed Jun. 16, 2022, titled “PATIENT-SPECIFIC ANTERIOR PLATE IMPLANTS;”
U.S. application Ser. No. 17/851,487, filed Jun. 28, 2022, titled “PATIENT-SPECIFIC ADJUSTMENT OF SPINAL IMPLANTS, AND ASSOCIATED SYSTEMS AND METHODS;”
U.S. application Ser. No. 17/856,625, filed Jul. 1, 2022, titled “SPINAL IMPLANTS FOR MESH NETWORKS;”
U.S. application Ser. No. 17/867,621, filed Jul. 18, 2022, titled “PATIENT-SPECIFIC SACROILIAC IMPLANT, AND ASSOCIATED SYSTEMS AND METHODS;”
U.S. application Ser. No. 17/868,729, filed Jul. 19, 2022, titled “SYSTEMS FOR PREDICTING INTRAOPERATIVE PATIENT MOBILITY AND IDENTIFYING MOBILITY-RELATED SURGICAL STEPS;”
U.S. application Ser. No. 17/978,673, filed Nov. 1, 2022, titled “SPINAL IMPLANTS AND SURGICAL PROCEDURES WITH REDUCED SUBSIDENCE, AND ASSOCIATED SYSTEMS AND METHODS;”
U.S. application Ser. No. 17/978,746, filed Nov. 1, 2022, titled “PATIENT-SPECIFIC SPINAL INSTRUMENTS FOR IMPLANTING IMPLANTS AND DECOMPRESSION PROCEDURES;”
U.S. application Ser. No. 18/102,444, filed Jan. 27, 2023, titled “TECHNIQUES TO MAP THREE-DIMENSIONAL HUMAN ANATOMY DATA TO TWO-DIMENSIONAL HUMAN ANATOMY DATA;”
U.S. application Ser. No. 18/113,573, filed Feb. 24, 2023, titled “PATIENT-SPECIFIC IMPLANT DESIGN AND MANUFACTURING SYSTEM WITH A DIGITAL FILING CABINET;”
U.S. Application No. 63/401,429, filed Aug. 26, 2022, titled “SYSTEMS AND METHODS FOR GENERATING, DESIGNING, AND/OR MODELING MULTIPLE PATIENT-SPECIFIC SURGICAL PLANS;”
U.S. application Ser. No. 17/951,085, filed Sep. 22, 2022, titled “SYSTEMS FOR MANUFACTURING AND PRE-OPERATIVE INSPECTING OF PATIENT-SPECIFIC IMPLANTS;”
U.S. Application No. 63/420,279, filed Oct. 28, 2022, titled “SYSTEMS AND METHODS FOR SELECTING, REVIEWING, MODIFYING, AND/OR APPROVING SURGICAL PLANS;”
U.S. Application No. 63/387,009, filed Dec. 12, 2022, titled “PATIENT-SPECIFIC IMPLANT DESIGN AND MANUFACTURING SYSTEM WITH A REGULATORY AND REIMBURSEMENT MANAGER;”
U.S. Application No. 63/436,860, filed Jan. 3, 2023, titled “PATIENT-SPECIFIC SPINAL FUSION DEVICES AND ASSOCIATED SYSTEMS AND METHODS;”
U.S. Application No. 63/437,966, filed Jan. 9, 2023, titled “SYSTEM FOR EDGE CASE PATHOLOGY IDENTIFICATION AND IMPLANT MANUFACTURING;” and
U.S. Application No. 63/437,975, filed Jan. 9, 2023, titled “SYSTEM FOR MODELING PATIENT SPINAL CHANGES.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

The ranges disclosed herein also encompass any and all overlap, sub-ranges, and combinations thereof. Language such as “up to,” “at least,” “greater than,” “less than,” “between,” or the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. A computer-implemented method for providing patient-specific medical care to a patient, the method comprising:
receiving patient data;
based on the patient data, generating a multi-stage surgical plan for treating the patient's spine and/or spinal region, wherein generating the multi-stage surgical plan includes:
determining a first treatment stage having a first surgical procedure, a first target location for the first surgical procedure, and a first period for performing the first surgical procedure, and
determining a second treatment stage having a second surgical procedure, a second target location for the second surgical procedure, and a second period for performing the second surgical procedure,
wherein the second treatment stage is based at least in part on a predicted outcome of the first treatment stage, and
wherein the second treatment stage is spaced apart in time from the first treatment stage; and
transmitting the multi-stage surgical plan for surgeon review.

2. The computer-implanted method of claim 1 wherein the second surgical procedure is a same type of procedure as the first surgical procedure.

3. The computer-implemented method of claim 2 wherein the first surgical procedure is a first spinal fusion procedure, and wherein the second surgical procedure is a second spinal fusion procedure.

4. The computer-implemented method of claim 1 wherein the first surgical procedure is a first interbody procedure, and wherein the second surgical procedure is a procedure to provide fixation.

5. The computer-implemented method of claim 1 wherein the first surgical procedure is a different type of procedure than the second surgical procedure.

6. The computer-implemented method of claim 1 wherein the first target location and the second target location are different.

7. The computer-implemented method of claim 6 wherein the first target location includes a first range of vertebrae and the second target location includes a second range of vertebrae.

8. The computer-implemented method of claim 7 wherein the first range of vertebrae overlap with the second range of vertebrae.

9. The computer-implemented method of claim 7 wherein the first range of vertebrae are spaced apart from the second range of vertebrae by at least one vertebra not included in the first range or the second range.

10. The computer-implemented method of claim 1 wherein the first surgical procedure includes an anterior approach and the second surgical procedure includes a posterior approach.

11. The computer-implemented method of claim 1 wherein the first surgical procedure includes a lateral approach and the second surgical procedure includes a posterior approach.

12. The computer-implemented method of claim 1 wherein the second period for performing the second surgical procedure is set based on a fixed duration from the first period for performing the first surgical procedure.

13. The computer-implemented method of claim 1 wherein the second period for performing the second surgical procedure is set based on a patient metric crossing a predefined threshold.

14. The computer-implemented method of claim 1 wherein the second treatment stage is spaced apart from the first treatment stage by at least about 6 months.

15. The computer-implemented method of claim 1 wherein the second treatment stage is spaced apart from the first treatment stage by at least about 1 year.

16. The computer-implemented method of claim 1 wherein the second treatment stage is spaced apart from the first treatment stage by at least about 3 years.

17. The computer-implemented method of claim 1 wherein generating the multi-stage surgical plan further comprising:
simulating an outcome of the first treatment stage, wherein simulating the outcome of the first treatment stage includes generating a first virtual model of predicted patient anatomy if the first treatment stage were performed; and
simulating an outcome of the second treatment stage, wherein simulating the outcome of the second treatment stage includes generating a second virtual model of predicted patient anatomy if the second treatment stage were performed.

18. The computer-implemented method of claim 17, further comprising determining an order for performing the first treatment stage and the second treatment stage based at least in part on the simulated outcomes.

19. The computer-implemented method of claim 17 wherein simulating the outcome of the first treatment stage include simulating an outcome under a variety of patient conditions, and wherein the first surgical procedure, the first target location, and/or the first period are determined based in part on the simulated outcome.

20. The computer-implemented method of claim 19 wherein the variety of patient conditions include different values for at least one of patient weight, patient BMI, or patient disability score.

21. The computer-implemented method of claim 1, further comprising:
generating a first virtual model of patient anatomy, the first virtual model associated with the first treatment stage; and
generating a second virtual model of patient anatomy, the second virtual model associated with the second treatment stage,
wherein the second virtual model incudes a predicted correction to patient anatomy to occur during first treatment phase.

22. The computer-implemented method of claim 1, further comprising simulating a change in patient anatomy over time between the first treatment stage and the second treatment stage.

23. The computer-implemented method of claim 22 wherein transmitting the multi-stage surgical plan for review includes transmitting simulated changes in the patient anatomy at discrete intervals between the first treatment stage and the second treatment stage.

24. The computer-implemented method of claim 22 wherein the simulated changes are shown using virtual models of patient anatomy.

25. The computer-implemented method of claim 1 wherein determining the second treatment stage includes:
determining a first alternative second treatment stage and a second alternative second treatment stage different than the first alternative second treatment stage; and
generating one or more selection criteria for selecting between the first alternative second treatment stage and the second alternative second treatment stage.

26. The computer-implemented method of claim 25 wherein the one or more selection criteria include patient metric thresholds following the first treatment stage.

27. The computer-implemented method of claim 1, further comprising:
defining a successful outcome for each treatment stage, the successful outcome 7 calculating a probability of achieving the successful outcome for reach treatment stage.

28. A non-transitory computer readable medium storing computer-executable instructions for generating multi-stage surgical plans using a computing system, wherein the instructions, when executed, cause the computing system to:
receive patient data;
based on the patient data, generate a multi-stage surgical plan for treating the patient's spine and/or spinal region, wherein the multi-stage surgical plan includes:
a first treatment stage having a first surgical procedure, a first target location for the first surgical procedure, and a first period for performing the first surgical procedure, and
a second treatment stage having a second surgical procedure, a second target location for the second surgical procedure, and a second period for performing the second surgical procedure,
wherein the second treatment stage is based at least in part on a predicted response to the first treatment stage, and
wherein the second treatment stage is spaced apart in time from the first treatment stage; and
transmit the multi-stage surgical plan for surgeon review.

29. The non-transitory computer readable medium of claim 28, wherein the instructions, when executed, further cause the computing system to:
simulate an outcome of the first treatment stage using a first virtual model; and
simulate an outcome of the second treatment stage using a second virtual model.

30. The computer-implemented method of claim 29 wherein the instructions, when executed, further cause the computing system to modify at least one of the first treatment stage or the second treatment stage based at least in part on the simulated outcome for the first treatment stage of the second treatment stage.

31. The computer-implemented method of claim 29 wherein the simulations include predicted patient metrics associated with performing the first treatment stage and the second treatment stage.

32. The computer-implemented method of claim 29 wherein a time between the first treatment stage and the second treatment stage is identified based on the simulated outcome for the first treatment stage and the second treatment stage.

33. A system for providing patient-specific medical treatment, the system comprising:
one or more processors; and
a memory storing instructions that, when executed by the one or more processors, case the system to perform operations comprising:
based on the patient data, generate a multi-stage surgical plan for treating the patient's spine and/or spinal region, wherein the multi-stage surgical plan includes:
a first treatment stage having a first surgical procedure, a first target location for the first surgical procedure, and a first period for performing the first surgical procedure, and
a second treatment stage having a second surgical procedure, a second target location for the second surgical procedure, and a second period for performing the second surgical procedure,
wherein the second treatment stage is based at least in part on a predicted response to the first treatment stage, and
wherein the second treatment stage is spaced apart in time from the first treatment; and
transmit the multi-stage surgical plan for surgeon review.

34. The system of claim 33 wherein the operation of generating the multi-stage surgical plan is performed at least in part by a trained machine learning model.

35. The system of claim 34 wherein the trained machine learning model compares the patient data to reference patient data to determine one or more aspects of the multi-stage surgical plan.

36. The system of claim 33 wherein the first treatment stage and the second treatment stage are spaced apart in time by at least 6 months.

37. The system of claim 33 wherein the operations further comprise:
generate a first virtual model showing predicted patient anatomy following the first treatment stage; and
generate a second virtual model showing predicted patient anatomy following the second treatment stage,
wherein the first virtual model and the second virtual model are transmitted with the multi-stage surgical plan for surgeon review.
